(19) United States
(12) Patent Application Publication
(10) Pub. No.: US 2024/0350446 A1
(43) Pub. Date: Oct. 24, 2024

(54) CAROTENOID SOLUTIONS AND USES THEREOF

(71) Applicant: L.E.A.F. HOLDINGS GROUP LLC, Gulph Mills, PA (US)

(72) Inventors: Clet NIYIKIZA, Gulph Mills, PA (US); Victor Mandla MOYO, Ringoes, NJ (US); Zhaohua Richard HUANG, Gulph Mills, PA (US)

(21) Appl. No.: 17/917,899

(22) PCT Filed: Apr. 9, 2021
(86) PCT No.: PCT/US2021/026513

§ 371 Date: Oct. 7, 2022

Related U.S. Application Data
(60) Provisional application No. 63/007,878, filed on Apr. 9, 2020.

(51) Int. Cl. A61K 31/202; A61K 9/127; A61K 45/06; A61K 47/10

(52) U.S. Cl. CPC A61K 31/202; A61K 9/127; A61K 45/06; A61K 47/10

(57) ABSTRACT

Provided herein are aqueous solutions and pharmaceutical compositions comprising ionizable carotenoids such as trans-crocetin. The provided compositions have uses in treating diseases, disorders and conditions associated with, but not limited to, infection, ARDS, endotoxemia, inflammation, sepsis, ischemia, hypoxia, shock, stroke, lung injury, wound healing, traumatic injury, reperfusion injury, cardiovascular disease, kidney disease, liver disease, inflammatory disease, metabolic disease, pulmonary disorders, blood related disorders and hyperproliferative diseases such as cancer. Methods of making, and using the aqueous solutions and pharmaceutical compositions are also provided.

Carotenoids are a class of natural lipid-soluble pigments found principally in plants where they function as accessory pigments and impart protection of tissue through their ability to quench singlet oxygen and free radical species. Carotenoids are known to have antioxidant properties and consequently, provide numerous beneficial health effects including reducing the potential risks of cardiovascular diseases, cancers, and slowing and/or reversing the degenerative effects of aging on various human physiological activities. However, carotenoids are typically very lipophilic compounds and the clinical use of many carotenoids is limited by their instability and low bioavailability.

Crocetin is a carotenoid with antioxidative properties that is sparingly soluble in water. Chemically, crocetin is a 20-carbon apocarotenoid molecule containing seven double bonds and a carboxylic acid group at each end. The administration of trans crocetin (free acid), and its salt sodium trans crocetinate in free form (e.g., unencapsulated) pharmaceutical formulations has been reported to offer promise in the treatment for conditions caused by hypoxia, ischemia, and other medical conditions. However, neither has demonstrated clinical therapeutic efficacy. This is partly due to the fact that formulations of trans crocetin and its sodium salt, sodium trans crocetinate, have been to date limited by instability, low bioavailability and short half-life.

In view of the health benefits conferred by carotenoids and the low bioavailability and instability outlined above, there is a need for providing aqueous solutions and pharmaceutical compositions comprising carotenoids with improved bioavailability and stability. The provided compositions and methods address the shortcomings of carotenoids described above. These compositions and methods will further help overcome the limitations of current therapeutic approaches to disease states linked to endotoxemia and hypoxia as well as other unmet medical needs. The compositions have applications as single agents and in combination with other therapies.

### BRIEF SUMMARY

The disclosure provides aqueous solutions and pharmaceutical compositions comprising carotenoids, including liposomes that encapsulate carotenoids such as trans crocetin, trans norbixin, and salts thereof. The provided compositions have uses in treating diseases and disorders and conditions associated with, but not limited to, infection, inflammation, sepsis, ischemia, hypoxia, shock, stroke, injury, cardiovascular disease, renal disease, liver disease, inflammatory disease, metabolic disease, pulmonary disease, neurodegenerative disease, disease of the immune system, and hyperproliferative diseases such as cancer. Methods of making, delivering, and using the aqueous solutions and pharmaceutical compositions are also provided, as are kits containing the compositions.

The disclosed aqueous solutions and pharmaceutical compositions provide for the enhanced delivery of carotenoids including ionizable Polyene Carotenoids such as trans-crocetin, with poor pharmacokinetics and biodistribution. The disclosure also provides liposome compositions that display high encapsulation efficiencies (>98%), high drug-to-lipid ratios, and/or enhanced drug retention. The provided aqueous solutions and pharmaceutical compositions have uses in treating diseases and disorders and conditions associated with, but not limited to, infection, inflammation, sepsis, ischemia, hypoxia, anemia, trauma, injury, stroke, shock, diabetes, wound healing, injury (e.g., reperfusion injury, neural injury, renal injury, livery injury and lung injury), and hyperproliferative diseases such as cancer, as well as conditions associated with the treatment of these diseases and disorders (e.g., anemia, neutropenia and immunosuppression). Methods of making, delivering, and using the compositions are also provided.

In some embodiments, the disclosure provides:
[1] an aqueous solution comprising an ionizable carotenoid salt having the formula:

Q-Polyene Carotenoid-Q, wherein,
the Polyene Carotenoid comprises
   (a) 3, 4, 5, 6, 7, 8, 9, 10, 3-5, 6-8, 9-10, or more than 9, conjugated double bonds,
   (b) methyl or low alkyl (C2-C3) substitutions, and
   (c) 1, 2, 3, or more than 3, ionizable groups, and
   Q is a monovalent counterion; and polyethylene glycol having a molecular weight of 200-700 Da;
[2] an aqueous solution comprising an ionizable carotenoid salt having the formula:

Q-R₁-Polyene Carotenoid-R₂-Q, wherein,
the Polyene Carotenoid comprises
   (a) 3, 4, 5, 6, 7, 8, 9, 10, 3-5, 6-8, 9-10, or more than 9, conjugated double bonds,
   (b) methyl or low alkyl (C2-C3) substitutions, and
   (c) 1, 2, 3, or more than 3, ionizable groups;
   R₁ and R₂ are ionizable groups (e.g., the same ionizable group or different ionizable groups), and
   Q is a monovalent counterion; and polyethylene glycol having a molecular weight of 200-700 Da;
[3] the aqueous solution of [1] or [2], wherein the Polyene Carotenoid comprises 1, 2, 3, or more than 3, anionic ionizable groups;
[4] the aqueous solution of [3], wherein the Polyene Carotenoid comprises at least one anionic ionizable group selected from: a carboxylic group, a sulfonate group, a sulfate group, a phosphonate, a phosphate group, and a hydroxamate group;
[5] the aqueous solution of [1] or [2], wherein the Polyene Carotenoid comprises 1, 2, 3, or more than 3, cationic ionizable groups (e.g., a primary, secondary, or tertiary amine group, a quaternary ammonium group, a choline group, a guanidine group, or an imidazole group);
[6] an aqueous solution comprising an ionizable carotenoid salt having the formula:

Q-trans-crocetin-Q,
wherein,
Q is a monovalent cation counterion, and polyethylene glycol having a molecular weight of 200-700 Da;
[7] an aqueous solution comprising an ionizable carotenoid salt having the formula:

Q-trans-norbixin-Q,
wherein,
Q is a monovalent cation counterion; and polyethylene glycol having a molecular weight of 200-700 Da;
[8] the aqueous solution according to any one of [1]-[7], wherein the ionizable carotenoid salt concentration is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, or at least 10 mg/mL;
[9] the aqueous solution according to any one of [1]-[8], wherein the ionizable carotenoid salt concentration is 0.01 mg/mL to 30 mg/mL, 0.05 mg/mL to 25 mg/mL, 0.075 mg/mL to 20 mg/mL, 0.1 mg/mL to 15 mg/mL, 0.5 mg/mL to 10 mg/mL, 1 mg/mL to 8 mg/mL, 1.5 mg/mL to 6 mg/mL, or 2 mg/mL to 5 mg/mL (e.g., 2 mg/mL, 3 mg/ml, 4 mg/mL, or 5 mg/mL);
[10] the aqueous solution according to any one of [1]-[9], wherein the monovalent counterion (Q) is NH₄⁺, Na⁺, Li⁺, K⁺ or a monovalent organic cation such as protonated amine;
[11] the aqueous solution according to any one of [1]-[10], wherein the monovalent counterion (Q) is Na⁺;
[12] the aqueous solution according to any one of [1]-[11], which comprises sodium trans-crocetinate (STC);
[13] the aqueous solution according to any one of [1]-[11], which comprises potassium trans-crocetinate (KTC), sodium trans-norbixinate (MTN), or potassium trans-norbixinate (KTN);
[14] the aqueous solution according to any one of [1]-[13], wherein the PEG has an average molecular weight between 200-700 Da, 200-600 Da, 300-500 Da, or 350-450 Da, (e.g., 400 Da);
[15] the aqueous solution according to any one of [1]-[14], wherein the PEG is PEG-200, PEG-300, PEG-400, PEG-500, or PEG-600;
[16] the aqueous solution according to any one of [1]-[15], which comprises PEG-400;
[17] the aqueous solution according to any one of [1]-[16], wherein the PEG concentration is 0.01% to 40%, 0.05% to 35%, 0.1% to 20%, 0.5% to 15%, 1% to 10%, 2% to 9%, 3% to 8%, or 5% to 7% (w/w);
[18] the aqueous solution according to any one of [1]-[17], wherein the PEG has an average molecular weight between 200-600 Da and the ionizable carotenoid salt to PEG ratio is 1:1-300; 1:1-100; 1-200; 1:1-50; 1:1-40, 1:5-30; or 1:10-25 (e.g., 1:20) (w/w);
[19] the aqueous solution according to any one of [1]-[18], wherein the PEG has an average molecular weight of about 400 Da and the ionizable carotenoid salt to PEG ratio is 1:1-300; 1:1-100; 1-200; 1:1-50; 1:1-40, 1:5-30; or 1:10-25 (e.g., 1:20) (w/w);
[20] the aqueous solution according to any one of [1]-[19], wherein the ionizable carotenoid salt is trans-crocetin (e.g., TSC), the PEG has an average molecular weight between 200-600 Da and the trans-crocetin to PEG ratio is 1:1-300; 1:1-100; 1-200; 1:1-50; 1:1-40, 1:5-30; or 1:10-25 (e.g., 1:20) (w/w);
[21] the aqueous solution of [20], wherein the PEG has an average molecular weight of about 400 Da and the ionizable carotenoid to PEG ratio is 1:1-300; 1:1-100; 1-200; 1:1-50; 1:1-40, 1:5-30; or 1:10-25 (e.g., 1:20) (w/w);
[22] the aqueous solution according to any one of [1]-[21], which has a pH of 6-10, 7.5-9.5, or 8-9 (e.g., pH 8.5), or any range therein between;
[23] the aqueous solution according to any one of [1]-[22], which comprises a buffer having a pKA within 1 unit or within 0.5 units of the pH of the solution at a concentration of 1-200 mM, 1-100 mM, 1-80 mM, or any range therein between;
[24] the aqueous solution according to any one of [1]-[23], which comprises a buffer selected from: glycine, gly-gly, sodium bicarbonate, sodium phosphate, tricine, bicine, EPPS (HEPPS), HEPBS, TABS, AMPD, or sodium borate (e.g., glycine, gly-gly, or sodium bicarbonate);
[25] the aqueous solution according to any one of [1]-[21], which further comprises a tonicity controlling agent such as at least one tonicity controlling agent selected from sodium chloride, mannitol, sorbitol, xylitol, dextrose, maltose, glucose, lactose and sucrose (e.g., sucrose or sodium chloride);
[26] the aqueous solution of [25], wherein the concentration of the tonicity controlling agent (e.g., sucrose or NaCl) is 0.05 mM to 100 mM, 0.75 mM to 75 mM, 1 mM to 50 mM, 5 mM to 40 mM, 7 mM to 30 mM, or 10 mM to 20 mM;
[27] the aqueous solution according to any one of [1]-[26], wherein the ionizable carotenoid salt concentration is 0.01 mg/mL to 30 mg/mL, the PEG has an average molecular weight between 200-700 Da and the PEG concentration is 0.05% to 35% (w/w);
[28] the aqueous solution of [27], wherein the ionizable carotenoid salt concentration is 0.1 mg/mL to 15 mg/mL, the PEG has an average molecular weight of 200-600 Da and the PEG concentration is 1% to 10% (e.g., 2% to 7%) (w/w);
[29] the aqueous solution according to any one of [1]-[28], wherein the ionizable carotenoid salt is trans-crocetin (e.g., TSC) at a concentration of 1 mg/mL to 5 mg/mL (e.g., 1 mg/mL, 2 mg/mL, 3 mg/mL, 4 mg/mL, or 5 mg/mL), the PEG has an average molecular weight between 200-600 Da and the PEG concentration is 1% to 10% (e.g., 5% to 7%) (w/w);
[30] the aqueous solution of [29], wherein the trans-crocetin (e.g., TSC) concentration is 1 mg/mL to 5 mg/mL (e.g., 1 mg/mL, 2 mg/mL, 3 mg/mL, 4 mg/mL, or 5 mg/mL), the PEG has an average molecular weight of about 400 Da (e.g., PEG-400) and the PEG concentration is 5% to 7% (w/w);
[31] the aqueous solution according to any one of [27]-[30], which further comprises a tonicity controlling agent;
[32] the aqueous solution according to any one of [27]-[30], wherein the tonicity controlling agent is sucrose or NaCl;
[33] the aqueous solution of [31] or [32], wherein the tonicity controlling agent (e.g., sucrose or NaCl) concentration is 0.05% to 20%, 0.1% to 15%, 0.5% to 10%, 0.75% to 8%, or 1% to 6% (w/w);
[34] the aqueous solution according to any one of [1]-[33], which further comprises a liposome encapsulating an ionizable carotenoid salt having the formula:

Z-Polyene Carotenoid-Z, wherein,
the Polyene Carotenoid comprises
   (a) 3, 4, 5, 6, 7, 8, 9, 10, 3-5, 6-8, 9-10, or more than 9, conjugated double bonds,
   (b) methyl or low alkyl (C2-C3) substitutions, and
   (c) 1, 2, 3, or more than 3, ionizable groups; and
   Z is a multivalent counterion;
[35] the aqueous solution according to any one of [1]-[33], which further comprises a liposome encapsulating an ionizable carotenoid salt having the formula:

Z—R₁-Polyene Carotenoid-R₂—Z, wherein,
the Polyene Carotenoid comprises
   (a) 3, 4, 5, 6, 7, 8, 9, 10, 3-5, 6-8, 9-10, or more than 9, conjugated double bonds,
   (b) methyl or low alkyl (C2-C3) substitutions, and
   (c) 1, 2, 3, or more than 3, ionizable groups;
   R₁ and R₂ are ionizable groups (e.g., the same ionizable group or different ionizable groups; and
   Z is a multivalent counterion;
[36] the aqueous solution of [34] or [35], wherein the liposome encapsulated ionizable carotenoid salt comprises 1, 2, 3, or more than 3, anionic ionizable groups;
[37] the aqueous solution of [36], wherein the liposome encapsulated ionizable carotenoid salt comprises at least one anionic ionizable group selected from: a carboxylic group, a sulfonate group, a sulfate group, a phosphonate, a phosphate group, and a hydroxamate group.
[38] the aqueous solution of [34] or [35], wherein the liposome encapsulated ionizable carotenoid salt comprises 1, 2, 3, or more than 3, cationic ionizable groups (e.g., a primary, secondary, or tertiary amine group, a quaternary ammonium group, a choline group, a guanidine group, or an imidazole group);
[39] the aqueous solution of [34] or [35], wherein the liposome encapsulated ionizable carotenoid salt has the formula:

Z-trans-crocetin-Z, wherein,
Z is a multivalent cation counterion;
[40] the aqueous solution of [34] or [35], wherein the liposome encapsulated ionizable carotenoid salt has the formula:

Z-norbixin-Z, wherein,
Z is a multivalent cation counterion;
[41] the aqueous solution according to any one of [34]-[40], wherein the multivalent counterion (Z) is a bivalent cation (e.g., a bivalent cation such as a bivalent metal cation or a bivalent organic cation, or a trivalent cation such as Fe³⁺);
[42] the aqueous solution according to any one of [34] or [41], wherein the multivalent counterion (Z) is at least one bivalent cation selected from Ca²⁺, Mg²⁺, Zn²⁺, Cu²⁺, Co²⁺, and Fe²⁺, a bivalent organic cation such as protonated diamine, or a trivalent cation such as Fe³⁺;
[43] the aqueous solution of [34] or [35], wherein the liposome encapsulates calcium trans-crocetinate (CTC);
[44] the aqueous solution of [34] or [35], wherein the liposome encapsulates magnesium trans-crocetinate (MTC), magnesium trans-crocetinate, magnesium trans-norbixinate (MTN) or calcium trans-norbixinate (CTN);
[46] a pharmaceutical composition comprising the aqueous solution according to any one of [1]-[45], [98], [99], [106], or [107];
[47] the pharmaceutical composition of [46], for use in the treatment or prevention of a disease or a condition in a subject;
[48] use of the pharmaceutical composition of [46] or [47] in the manufacture of a medicament for the treatment or prevention of a disease or condition in a subject;
[49] a method for treating or preventing a disease or condition in a subject needing such treatment or prevention, the method comprising administering an effective amount of the pharmaceutical composition of [46] or [47] to the subject;
[50] the pharmaceutical composition of [47], use of [48], or method of [49], wherein the disease or condition is characterized by ischemia (e.g., tissue hypoperfusion) or hypoxia;
[51] the pharmaceutical composition, use, or method, of [50], wherein the disease or condition characterized by ischemia or hypoxia is tissue hypoperfusion, ischemic-reperfusion injury, transient cerebral ischemia, cerebral ischemia-reperfusion, ischemic stroke, hemorrhagic stroke, traumatic brain injury, migraine (e.g., a chronic migraine or severe migraine disorder), gastrointestinal ischemia, kidney disease, pulmonary embolism, acute respiratory failure, neonatal respiratory distress syndrome, an obstetric emergency to reduce perinatal comorbidity (such as, pre/eclampsia and conditions that lead to cerebral palsy), myocardial infarction, acute limb or mesenteric ischemia, cardiac cirrhosis, chronic peripheral vascular disease, congestive heart failure, atherosclerotic stenosis, anemia, thrombosis, or embolism, wherein the subject has experienced a traumatic injury, or wherein the subject is undergoing or will undergo surgery;
[52] the pharmaceutical composition of [47], use of [48], or method of [49], wherein the disease or condition is a lung disease or a condition associated with a lung disease;
[53] the pharmaceutical composition, use, or method of [52], wherein the disease or condition is pulmonary fibrosis, pulmonary hemorrhage, lung injury, lung cancer, chronic obstructive pulmonary disease (COPD), or another respiratory disorder;
[54] the pharmaceutical composition of [47], use of [48], or method of [49], wherein the disease or condition is acute respiratory distress syndrome (ARDS);
[55] the pharmaceutical composition of [47], use of [48], or method of [49], wherein the disease or condition is a cardiovascular disease or a condition associated with a cardiovascular disease;
[56] the pharmaceutical composition, use, or method of [55], wherein the disease or condition is a coronary artery disease such as myocardial infarction, sudden cardiac death, cardiorespiratory arrest, hypertension, pulmonary arterial hypertension, atherosclerosis, Takayasu's arthritis, and post-cardiac arrest syndrome (PCAS), chronic venous insufficiency, heart disease, congestive heart failure, occlusive arterial disease, Raynaud's disease, peripheral vascular disease, or another vasculopathy such as Buerger's disease, or a chronic skin ulcer;
[57] the pharmaceutical composition of [47], use of [48], or method of [49], wherein the disease or condition is a heart attack or stroke, or a condition associated with a heart attack or stroke (e.g., an ischemic stroke or hemorrhagic stroke);
[58] the pharmaceutical composition of [47], use of [48], or method of [49], wherein the disease or condition is associated with nitric oxide deficiency (e.g., sickle cell disease, paroxysmal nocturnal hemoglobinuria (PNH), a hemolytic anemia, a thalassemia, another red blood cell disorder, a purpura such as thrombotic thrombocytic purpura (TTP), hemolytic uremic syndrome (HUS), idiopathic thrombocytopenia (ITP), another platelet disorder, a coagulation abnormality such as disseminated intravascular coagulopathy (DIC), purpura fulminans, heparin induced thrombocytopenia (HIT), hyperleukocytosis, and or hyper viscosity syndrome);
[59] the pharmaceutical composition of [47], use of [48], or method of [49], wherein the disease or condition is shock or a condition associated with shock (e.g., cardiogenic shock, hypovolemic shock, septic shock, neurogenic shock, and anaphylactic shock);
[60] the pharmaceutical composition of [47], use of [48], or method of [49], wherein the disease or condition is an infection or a condition associated with an infection (e.g., hypoxia, tissue hypoperfusion, ischemia, ARDS, and sepsis);
[61] the pharmaceutical composition, use, or method, of [60], wherein the disease or condition is associated with a bacterial infection (e.g., an P. aeruginosa infection, S. aureus infection (e.g., MRSA), or an enterococcal infection (e.g., VRE), such as hypoxia, tissue hypoperfusion, ischemia, and sepsis);
[62] the pharmaceutical composition, use, or method, of [60], wherein the disease or condition is associated with a viral infection (e.g., hypoxia, tissue hypoperfusion, ischemia, sepsis, and ARDS;
[63] the pharmaceutical composition, use, or method, of [62], wherein the disease or condition is associated with a coronavirus infection (e.g., COVID-19 and ARDS);
[64] the pharmaceutical composition, use, or method, of [62], wherein the disease or condition is associated with an Ebola, Dengue or Marburg infection (e.g., influenza, measles, and a viral hemorrhagic fever);
[65] the pharmaceutical composition, use, or method, of [60], wherein the disease or condition is associated with a fungal infection (e.g., a candidiasis infection such as invasive candidiasis);
[66] the pharmaceutical composition, use, or method, of [60], wherein the disease or condition is a parasitic infection or a condition associated with a parasitic infection such as malaria (e.g., cerebral malaria, severe anemia, acidosis, acute kidney failure, ischemia, tissue hypoperfusion and ARDS), Schistosomiasis, and human African trypanosomiasis;
[67] the pharmaceutical composition of [47], use of [48], or method of [49], wherein the disease or condition is endotoxemia or a condition associated with endotoxemia (e.g., low grade endotoxemic disease, or endotoxemia associated with a condition such as periodontal disease (e.g., periodontitis or inflammation of the gums), chronic alcoholism, chronic smoking, transplantation, neonatal necrotizing enterocolitis, or a neonatal ear infection);
[68] the pharmaceutical composition of [47], use of [48], or method of [49], wherein the disease or condition is bacteremia;
[69] the pharmaceutical composition of [47], use of [48], or method of [49], wherein the disease or condition is sepsis; or a condition associated with sepsis, or wherein the subject is at risk of developing sepsis or a condition associated with sepsis;
[70] the pharmaceutical composition of [47], use of [48], or method of [49], wherein the disease or condition is inflammation or a condition associated with inflammation;
[71] the pharmaceutical composition, use, or method of [70], wherein the disease or condition is systemic inflammation (e.g., systemic inflammatory response syndrome (SIRS)), low-grade inflammation, acute inflammation, or a chronic inflammatory disease;
[72] the pharmaceutical composition of [47], use of [48], or method of [49], wherein the disease or condition is an autoimmune disorder or a condition associated with an autoimmune disorder (e.g., psoriasis, cystic fibrosis, and rheumatoid arthritis);
[73] the pharmaceutical composition of [47], use of [48], or method of [49], wherein the disease or condition is sclerosis or a condition associated with sclerosis (e.g., systemic sclerosis);
[74] the pharmaceutical composition of [47], use of [48], or method of [49], wherein the disease or condition is inflammatory bowel disease or a condition associated with inflammatory bowel disease (e.g., Crohn's disease and ulcerative colitis);
[75] the pharmaceutical composition of [47], use of [48], or method of [49], wherein the disease or condition is a metabolic disease or a condition associated with a metabolic disease;
[76] the pharmaceutical composition of [47], use of [48], or method of [49], wherein the disease or condition is insulin resistance or a condition associated with insulin resistance;
[77] the pharmaceutical composition of [47], use of [48], or method of [49], wherein the disease or condition is diabetes or a condition associated with diabetes such as gangrene, diabetic necrosis, diabetic neuropathy, diabetic vascular disease (e.g., microvascular disease such as retinopathy and nephropathy, and diabetic ulcers);
[78] the pharmaceutical composition of [47], use of [48], or method of [49], wherein the disease or condition is type 2 diabetes or a condition associated with type 2 diabetes;
[79] the pharmaceutical composition of [47], use of [48], or method of [49], wherein the disease or condition is liver disease or a condition associated with a liver disease (e.g., cirrhosis, nonalcoholic fatty liver disease (NAFLD), non-alcoholic steatohepatitis (NASH); alcoholic liver disease, acute liver injury, and cirrhosis of the liver);
[80] the pharmaceutical composition of [47], use of [48], or method of [49], wherein the disease or condition is kidney disease or a condition associated with kidney disease (e.g., lipopolysaccharide medication or toxin induced acute kidney injury (AKI) and end stage kidney disease);
[81] use of the pharmaceutical composition of [46] or [47] in the manufacture of a medicament for increasing the delivery of oxygen to the cells in a subject;
[82] a method of increasing the delivery of oxygen to the cells and tissues in a subject, the method comprising administering an effective amount of the pharmaceutical composition of [46] or [47] to the subject;
[83] the use of [81] or method of [82], wherein the subject is in need of increased oxygen delivery;
[84] the use of [81] or method of [82] or [83], wherein the pharmaceutical composition is administered to increase the physical or mental performance of the subject;
[85] use of the pharmaceutical composition of [46] or [47] in the manufacture of a medicament for reducing systemic levels of LPS, endotoxin and/or another trigger of systemic inflammation in a subject in a subject in a subject;
[86] a method of reducing systemic levels of LPS, endotoxin and/or another trigger of systemic inflammation in a subject in need thereof, the method comprising administering an effective amount of the pharmaceutical composition of [46] or [47] to the subject;
[87] the use or method according to any one of [47]-[86], wherein the age of the subject is 50 or older, 55 or older, 60 or older, 65 or older, 70 or older, 75 or older, or 80 or older;
[88] the use or method according to any one of [47]-[87], wherein the subject is immunocompromised;
[89] the use or method according to any one of [47]-[88], wherein the subject receives chemotherapy and/or is immune-suppressed (e.g., febrile neutropenic patients);
[90] the use or method according to any one of [47]-[89], wherein the subject is a burn victim;
[91] the use or method according to any one of [47]-[90], wherein the subject is critically ill;
[92] the use or method according to any one of [47]-[91], wherein the pharmaceutical composition is administered in combination therapy with another therapeutic agent;
[93] a method of preparing a loaded liposomal pharmaceutical composition comprising:
   (a) mixing the aqueous solution according to any one of [1]-[33] with a liposomal solution containing liposomes in a weak acid salt of a multivalent metal; and
   (b) maintaining the mixture of (a) for a sufficient time to load carotenoid into liposomes;
[94] the method of [93], wherein the weak acid is an organic acid (e.g., an organic acid selected from acetic acid, gluconic acid, tartaric acid, glutamic acid, citric acid, formic acid, and glycinic acid);
[95] the method of [93] or [94], wherein the multivalent metal is a bivalent metal (e.g., a bivalent metal selected from Ca²⁺, Mg²⁺, Zn²⁺, Cu²⁺, Co²⁺, and Fe²⁺), or a trivalent metal such as Fe³⁺;
[96] the method according to any of [93]-[95], wherein the weak acid is acetic acid and the bivalent metal is Ca²⁺ or Mg²⁺ (i.e., the weak acid salt of the bivalent metal is calcium acetate or magnesium acetate, respectively);
[97] the method according to any one of [93]-[96], which further comprises the step of heating and cooling of the drug loading mixture;
[98] an aqueous solution comprising a liposome prepared according to the method of any one of [93]-[97];
[99] the aqueous solution according to any one of [34]-[45], which comprises a liposome prepared according to the method of any one of [93]-[97];
[100] a pharmaceutical composition comprising a liposome prepared according to the method of any one of [93]-[97];
[101] a method of preparing pharmaceutical composition comprising a liposome encapsulating trans-crocetin, the method comprising:
   (a) preparing a liposomal solution comprising liposomes and a solution containing a weak acid salt of a monovalent metal;
   (b) adding the trans-crocetin to the liposomal solution; and
   (c) maintaining the trans-crocetin in the liposomal solution for sufficient time to load trans-crocetin into liposomes;
[102] the method of [102], wherein the weak acid is an organic acid (e.g., an organic acid selected from acetic acid, gluconic acid, tartaric acid, glutamic acid, citric acid, formic acid, and glycinic acid);
[103] the method of [101] or [102], wherein the multivalent metal is a bivalent metal (e.g., a bivalent metal selected from Ca²⁺, Mg²⁺, Zn²⁺, Cu²⁺, Co²⁺, and Fe²⁺), or a trivalent metal such as Fe³⁺;
[104] the method according to any of [101]-[103], wherein the weak acid is acetic acid and the bivalent metal is Ca²⁺ or Mg²⁺ (i.e., the weak acid salt of the bivalent metal is calcium acetate or magnesium acetate, respectively);
[105] the method according to any one of [101]-[104], which further comprises the step of heating and cooling of the drug loading mixture, diafiltration and sterile filtration;
[106] an aqueous solution comprising a liposome prepared according to the method of any one of [101]-[105];
[107] the aqueous solution according to any one of [34]-[45], which comprises a liposome prepared according to the method of any one of [101]-[105]; and/or
[108] a pharmaceutical composition comprising a liposome prepared according to the method of any one of [93]-[97];

Still other features and advantages of the aqueous solution and pharmaceutical compositions and methods described herein will become more apparent from the following detailed description when read in conjunction with the accompanying drawings.

### BRIEF DESCRIPTION OF THE DRAWINGS/FIGURES

FIG. 1A, FIG. 1B, FIG. 1C, and FIG. 1D, depict the exemplary ionizable Polyene Carotenoids of the provided aqueous solutions and pharmaceutical compositions.

FIG. 2 depicts an exemplary synthesis scheme for sodium trans-crocetinate.

FIG. 3 depicts the chemical structure sodium trans-crocetinate (molecular weight 372.37) and calcium trans-crocetinate (molecular weight 366.47).

FIG. 4 presents a survival at Day 5 after treatment with L4L-121 plus imipenem vs saline plus imipenem in a CLP model (L4L-121 Efficacy Study 1).

FIG. 5 presents a survival at Day 5 after treatment with L4L-121 plus imipenem vs saline plus imipenem in a CLP model (L4L-121 Efficacy Study 2).

FIG. 6 presents the treatment effect of L4L-121 vs saline on aspartate aminotransferase, creatinine, blood urea nitrogen, alanine aminotransferase, bilirubin, albumin and alkaline phosphatase (L4L-121 Efficacy Study 2). The horizontal dotted lines represent the upper and lower ranges of normal.

FIG. 7 presents the percent survival at Day 5 after treatment with L4L-121 plus imipenem vs saline plus imipenem in a CLP model (L4L-121 Efficacy Study 3).

FIG. 8 presents Treatment effect of LAL-121 vs saline on aspartate aminotransferase, creatinine, blood urea nitrogen, calcium, bilirubin, albumin, alkaline phosphatase and glucose. The horizontal dotted lines represent the upper and lower ranges of normal (L4L-121 Efficacy Study 3).

FIG. 9 presents the percent survival at Day 5 after treatment with L4L-121 plus imipenem vs saline plus imipenem in a CLP model (L4L-121 Efficacy Study 4).

FIG. 10 presents the treatment effect of different doses of L4L-121 on alanine aminotransferase, creatinine, blood urea nitrogen, calcium, bilirubin, albumin and alkaline phosphatase (L4L-121 Efficacy Study 4). The horizontal dotted lines represent the upper and lower ranges of normal.

### DETAILED DESCRIPTION

The Applicants have surprisingly discovered formulations containing aqueous solutions that improve the solubility, stability and bioavailability of the ionizable carotenoid salts such as sodium trans-crocetinate compared to conventional carotenoid formulations.

### Definitions

Unless otherwise defined, all technical and scientific terms used herein have the same meaning as commonly understood by one of ordinary skill in the art to which this disclosure pertains. Although methods and materials similar or equivalent to those described herein can be used in the practice or testing of the provided compositions, suitable methods and materials are described below. Each publication, patent application, patent, and other reference mentioned herein is herein incorporated by reference in its entirety. In case of conflict, the present specification, including definitions, will control. In addition, the materials, methods, and examples are illustrative only and are not intended to be limiting.

Other features and advantages of the disclosed compositions and methods will be apparent from the following disclosure, drawings, and claims.

It is understood that wherever embodiments, are described herein with the language “comprising” otherwise analogous embodiments, described in terms of “containing” “consisting of” and/or “consisting essentially of” are also provided. However, when used in the claims as transitional phrases, each should be interpreted separately and in the appropriate legal and factual context (e.g., in claims, the transitional phrase “comprising” is considered more of an open-ended phrase while “consisting of” is more exclusive and “consisting essentially of” achieves a middle ground).

As used herein, the singular form “a”, “an”, and “the”, include plural forms unless it is expressly stated or is unambiguously clear from the context that such is not intended. The singular form “a”, “an”, and “the” also includes the statistical mean composition, characteristics, or size of the particles in a population of particles (e.g., mean polyethylene glycol molecular weight mean liposome diameter, mean liposome zeta potential). The mean particle size and zeta potential of liposomes in a pharmaceutical composition can routinely be measured using methods known in the art, such as dynamic light scattering. The mean amount of a therapeutic agent in a nanoparticle composition may routinely be measured for example, using absorption spectroscopy (e.g., ultraviolet-visible spectroscopy).

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments, of the disclosure are described in terms of a Markush group or other grouping of alternatives, the disclosed composition or method encompasses not only the entire group listed as a whole, but also each member of the group individually and all possible subgroups of the main group, and also the main group absent one or more of the group members. The disclosed compositions and methods also envisage the explicit exclusion of one or more of any of the group members in the disclosed compositions or methods.

The term “liposome” refers to a closed vesicle having an internal phase (i.e., interior space (internal solution)) enclosed by lipid bilayer. A liposome can be a small single-membrane liposome such as a small unilamellar vesicle (SUV), large single-membrane liposome such as a large unilamellar vesicle (LUV), a still larger single-membrane liposome such as a giant unilamellar vesicle (GUV), a multilayer liposome having multiple concentric membranes (e.g., 2, 3, 4, 5, 6, 7, 8, 9, or 10), such as a multilamellar vesicle (MLV), or a liposome having multiple membranes that are irregular and not concentric such as a multivesicular vesicle (MVV). Liposomes and liposome formulations are well known in the art. Lipids which are capable of forming liposomes include all substances having fatty or fat-like properties. Lipids which can make up the lipids in the liposomes include without limitation, glycerides, glycerophospholipids, glycerophosphinolipids, glycerophos-phonolipids, sulfo-lipids, sphingolipids, phospholipids, isoprenolides, steroids, stearines, sterols, archeolipids, synthetic cationic lipids and carbohydrate containing lipids.

A “liposome composition” is a prepared composition comprising a liposome and the contents within the liposome, particularly including the lipids which form the liposome bilayer(s), compounds other than the lipids within the bi-layer(s) of the liposome, compounds within and associated with the aqueous interior(s) of the liposome, and compounds bound to or associated with the outer layer of the liposome. Thus, in addition to the lipids of the liposome, a liposome composition described herein suitably may include, but is not limited to, therapeutic agents, immunostimulating agents, vaccine antigens and adjuvants, excipients, carriers and buffering agents. In a preferred embodiment, such compounds are complementary to and/or are not significantly detrimental to the stability or AGP-incorporation efficiency of the liposome composition.

The term “counterion” refers to an anionic or cationic counterion. A “cationic counterion” is a positively charged atom or group associated with an anionic atom or group in order to maintain electronic neutrality. Exemplary cationic counterions include inorganic cations (e.g., metal cations (e.g., alkali metal cations, alkali earth metal cations, and transition metal cations)) and organic cations (e.g., ammonium cations, sulfonium cations, phosphonium cations, and pyridinium cations). An “anionic counterion” is a negatively charged atom or group associated with a cationic atom or group in order to maintain electronic neutrality. Exemplary anionic counterions include halide anions (e.g., F⁻, Cl⁻, Br⁻, and I⁻), NO3⁻, ClO4⁻OH⁻, H2PO4⁻², HSO4⁻, sulfonate anions (e.g., methansulfonate, trifluoromethane-sulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate anions (e.g., acetate, ethanoate, propanoate, benzoate, glycerate, lactate, tartrate, and glycolate). A counterion may be monovalent or multivalent (e.g., bivalent, trivalent, tetravalent, etc.).

The term “ionizable” refers to a compound containing at least one functional group that (a) bears a positive or negative charge (i.e., is “ionized”) and is therefore associated with a counterion of opposite charge, or (b) is electronically neutral but ionized at a higher or lower pH. Thus, ionizable compounds include quaternary ammonium salts as well as uncharged amines, and carboxylate moieties as well as uncharged carboxyl groups.

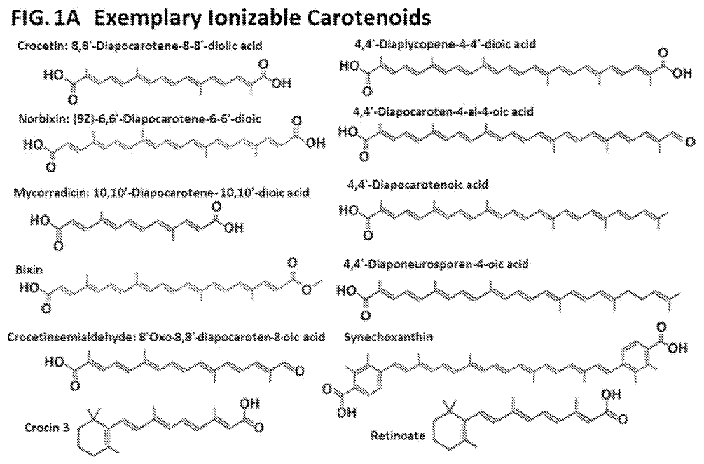

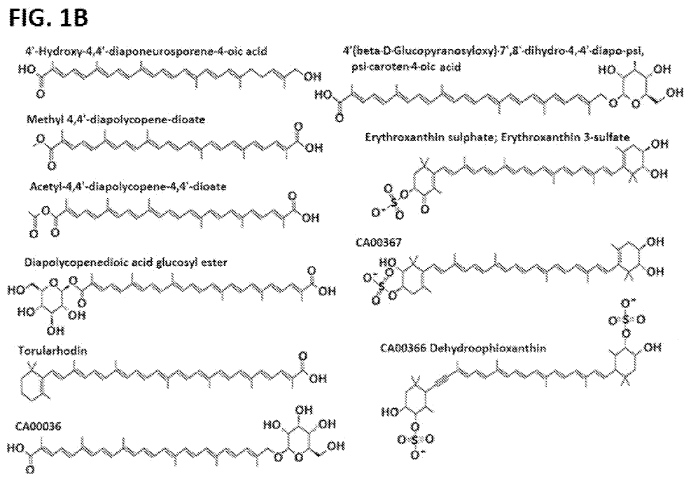

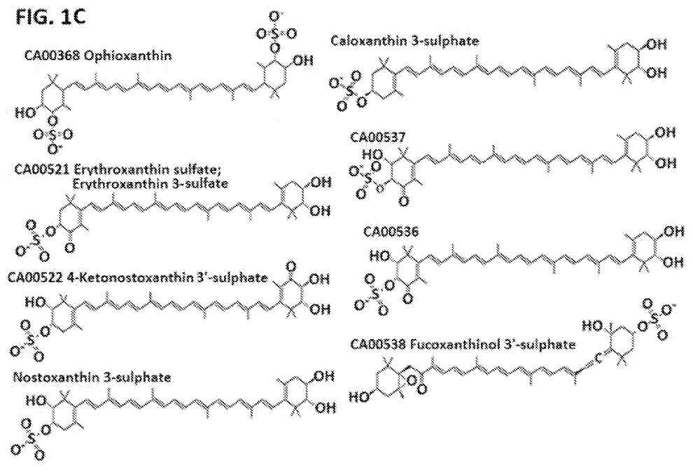

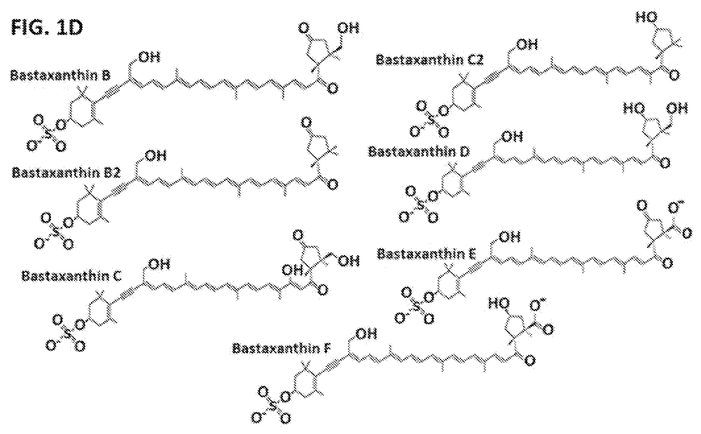

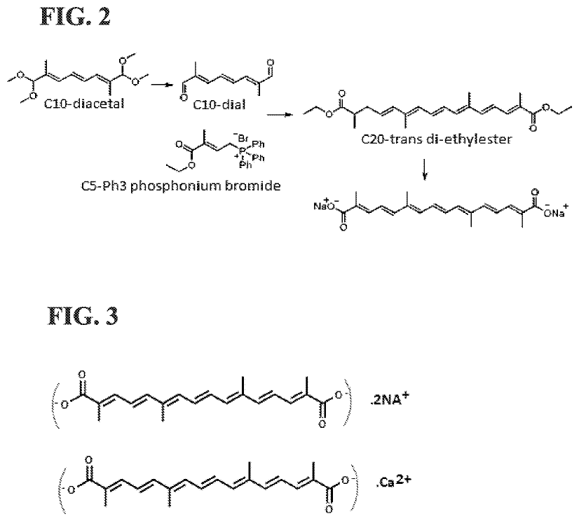

The term “carotenoid”, as used herein, refers to organic pigments which are structurally composed of a polyene hydrocarbon chain, and which may terminate in a ring. Carotenoids are divided into two classes, xanthophylls (which contain oxygen atoms) and carotenes (which contain no oxygen atoms). Non-limiting examples of carotenoids suitable for use in the provided compositions and methods are provided in FIG. 1A-FIG. 1D. Carotenoids with ionizable functional groups comprise naturally occurring carotenoid sulphates, carotenoid carboxylic acids/carboxylates, synthetic phosphates, blue carotenoid oxonium ions and blue carotenoproteins.

The term “Polyene Carotenoid” as used herein, refers to a carotenoid containing 3 or more conjugated double bonds, and methyl or low alkyl (C2-C3) substitutions.

The term “naturally occurring” refers to a compound or composition that occurs in nature, regardless of whether the compound or composition has been isolated from a natural source or chemically synthesized. Examples of naturally occurring carotenoid mono- and di-carboxylic acids include crocetin, norbixin, azafrin and neurosporaxanthin.

An “apocarotenoid” is a carotenoid degradation product in which the normal structure (e., C40) has been shortened by the removal of fragments from one or both ends. Examples of naturally occurring apocarotenoids include crocetin (C20), bixin (C25), Vitamin A, abscisic acid, mycorradicin and blumenin.

As used herein an “effective amount” refers to a dosage of an agent sufficient to provide a medically desirable result. The effective amount will vary with the desired outcome, the particular disease or condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. An “effective amount” can be determined empirically and in a routine manner, in relation to the stated purpose. In the case of cancer, the effective amount of an agent may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, duration of progression free survival (PFS), the response rates (RR), duration of response, and/or quality of life. In the case of a pulmonary disorder such as ARDs, COPD, sepsis or pulmonary inflammation, an effective amount of a agent may include include for example, stable or improving lung function, such as demonstrated by physical examination and respiratory rate normalization, improving pAO2/FiO2 ratio (P/F ratio), normalization of pCO₂, preventing need for intubation and mechanical ventilation, (for those mechanically ventilated) decreased number of ventilator days, decreased hospital length of stay, decreased intensive care unit length of stay, or a combination thereof.

The terms “hyperproliferative disorder”, “proliferative disease”, and “proliferative disorder”, are used interchangeably herein to pertain to an unwanted or uncontrolled cellular proliferation of excessive or abnormal cells which is undesired, such as, neoplastic or hyperplastic growth, whether in vitro or in vivo. In some embodiments, the proliferative disease is cancer or tumor disease (including benign or cancerous) and/or any metastases, wherever the cancer, tumor and/or the metastasis is located. In some embodiments, the proliferative disease is a benign or malignant tumor. In some embodiments, the proliferative disease is a non-cancerous disease. In some embodiments, the proliferative disease is a hyperproliferative condition such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

“Cancer,” “tumor,” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (metastasize) as well as any of a number of characteristic structural and/or molecular features. “Tumor,” as used herein refers to all neoplastic cell growth and proliferation, whether malignant or benign, and all pre-cancerous and cancerous cells and tissues. A “cancerous tumor,” or “malignant cell” is understood as a cell having specific structural properties, lacking differentiation and being capable of invasion and metastasis. A cancer that can be treated using a carotenoid pharmaceutical composition provided herein includes without limitation, a non-hematologic malignancy including such as for example, lung cancer, pancreatic cancer, breast cancer, ovarian cancer, prostate cancer, head and neck cancer, gastric cancer, gastrointestinal cancer, colorectal cancer, esophageal cancer, cervical cancer, liver cancer, kidney cancer, biliary duct cancer, gallbladder cancer, bladder cancer, sarcoma (e.g., osteosarcoma), brain cancer, central nervous system cancer, and melanoma; and a hematologic malignancy such as for example, a leukemia, a lymphoma and other B cell malignancies, myeloma and other plasma cell dysplasias or dyscrasias. Other types of cancer and tumors that may be treated using a trans-crocetin composition are described herein or otherwise known in the art. The terms “cancer,” “cancerous,” “cell proliferative disorder,” “proliferative disorder,” and “tumor” are not mutually exclusive as referred to herein.

“Ischemia” relates to a restriction in blood supply to tissues or organs (tissue hypoperfusion) causing a shortage of oxygen needed for cellular metabolism. The term “ischemia injury”, as used herein, relates to the damage due to a shortage of oxygen needed for cellular metabolism.

“Reperfusion” refers to the restoration of blood flow to ischemic tissue.

The term “ischemia/reperfusion injury”, also known as “ischemia/reperfusion damage” relates to organ or tissue damage caused when blood supply returns to the organ or tissue after a period of ischemia. The absence of oxygen and nutrients from blood during the ischemic period creates a condition in which the restoration of circulation results in inflammation and oxidative damage through the induction of oxidative stress rather than restoration of normal function. Oxidative stress associated with reperfusion may cause damage to the affected tissues or organs. Ischemia/reperfusion injury is characterized biochemically by a depletion of oxygen during an ischemic event followed by reoxygenation and the concomitant generation of reactive oxygen species during reperfusion. Examples of ischemia injury or ischemia/reperfusion injury include organ dysfunction (in the ischemic organ or in any other organ), infarct, inflammation (in the damaged organ or tissue), oxidative damage, mitochondrial membrane potential damage, apoptosis, reperfusion-related arrhythmia, cardiac stunning, cardiac lipotoxicity, ischemia-derived scar formation, and combinations thereof. In some embodiments, ischemia/reperfusion injury is assessed by using oxidative stress biochemical markers such as malondialdehyde (MDA), high-sensitivity troponin T (hs-TnT), high-sensitivity troponin T (hs-Tnl), creatin kinase myocardial band (CK-MB), and the inflammatory cytokines TNF-alpha IL-1 beta, IL-6, and IL-10.

“Organ dysfunction” refers to a condition wherein a particular organ does not perform its expected function. An organ dysfunction develops into organ failure if the normal homeostasis cannot be maintained without external clinical intervention. Methods to determine organ dysfunction are known in the art and include without limitation, monitorization and scores including sequential organ failure assessment (SOFA) score, multiple organ dysfunction (MOD) score and logistic organ dysfunction (LOD) score.

Terms such as “treating,” or “treatment,” or “to treat” refer to both (a) therapeutic measures that cure, slow down, attenuate, lessen symptoms of, and/or halt progression of a diagnosed pathologic condition or disorder and (b) prophylactic or preventative measures that prevent and/or slow the development of a targeted disease or condition. Thus, subjects in need of treatment include those already with the cancer, disorder or disease; those at risk of having the cancer or condition; and those in whom the infection or condition is to be prevented. Subjects are identified as “having or at risk of having” sepsis, an infectious disease, a disorder of the immune system, a metabolic disorder (e.g., diabetes), a hyperproliferative disease, or another disease or disorder referred to herein using well-known medical and diagnostic techniques. In certain embodiments, a subject is successfully “treated” according to the methods provided herein if the subject shows, e.g., total, partial, or transient amelioration or elimination of a symptom associated with the disease or condition (e.g., cancer and arthritis such as rheumatoid arthritis). In specific embodiments, the terms “treating,” or “treatment,” or “to treat” refer to the amelioration of at least one measurable physical parameter of a proliferative disorder, such as growth of a tumor, not necessarily discernible by the patient. In other embodiments, the terms “treating,” or “treatment,” or “to treat” refer to the inhibition of the progression of a proliferative disorder, either physically by, e.g., stabilization of a discernible symptom, physiologically by, e.g., stabilization of a physical parameter, or both. In other embodiments, the terms “treating,” or “treatment,” or “to treat” refer to the reduction or stabilization of tumor size, tumor cell proliferation or survival, or cancerous cell count. Treatment can be with a provided pharmaceutical composition disclosed herein (e.g., a liposomal trans-crocetinate) alone, or in combination with an additional therapeutic agent.

“Subject” and “patient,” and “animal” are used interchangeably and refer to mammals such as human patients and non-human primates, as well as experimental animals such as rabbits, rats, and mice, and other animals. Animals include all vertebrates, e.g., mammals and non-mammals, such as chickens, amphibians, and reptiles. “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and other members of the class Mammalia known in the art. In a particular embodiment, the patient is a human.

The term “elderly” refers to an aged subject, who has passed middle age. In one embodiment, an elderly mammalian subject is a subject that has survived more than two-thirds of the normal lifespan for that mammalian species. In a further embodiment, for humans, an aged or elderly subject is more than 65 years of age, such as a subject of more than 70, more than 75, more than 80 years of age. In yet another embodiment, for mice, an elderly mouse is from about 14 to about 18 months of age.

The term “pharmaceutically acceptable carrier” refers to an ingredient in a pharmaceutical formulation, other than an active ingredient, which is nontoxic to a subject. A pharmaceutically acceptable carrier includes, but is not limited to, a buffer, carrier, excipient, stabilizer, diluent, or preservative. Pharmaceutically acceptable carriers can include for example, one or more compatible solid or liquid filler, diluents or encapsulating substances which are suitable for administration to a human or other subject.

“Therapeutic agent”: the therapeutic agent or therapeutic agents used according to the disclosed compositions and methods can include any agent directed to treat a condition in a subject. Examples of therapeutic agents that may be suitable for use in accordance with the disclosed methods include vitamin C, thiamine, hydrocortisone or another corticosteroid (e.g., a glucocorticoid such as, cortisone, ethamethasoneb, prednisone, prednisolone, triamcinolone, dexamethasone and methylprednisolone; and mineralocorticoids such as fludrocortisonel), astaxanthin, abscisic acid, vitamin A, angiotensin II (e.g., GIAPREZA™), tissue plasminogen activator (tPA), an antimicrobial (e.g., antibiotic or chloroquin and its analogs) and an anti-inflammatory.

Additional examples of therapeutic agents that may be suitable for use in accordance with the disclosed methods include, without limitation, anti-restenosis, pro- or anti-proliferative, anti-neoplastic, antimitotic, anti-platelet, anticoagulant, antifibrin, antithrombin, cytostatic, antibiotic and other anti-infective agents, anti-enzymatic, anti-metabolic, angiogenic, cytoprotective, angiotensin converting enzyme (ACE) inhibiting, angiotensin II receptor antagonizing and/or cardioprotective agents. In general, any therapeutic agent known in the art can be used, including without limitation agents listed in the United States Pharmacopeia (U.S.P.), Goodman and Gilman's The Pharmacological Basis of Therapeutics, 10th Ed., McGraw Hill, 2001; Katzung, Ed., Basic and Clinical Pharmacology, McGraw-Hill/Appleton & Lange, 8th ed., Sep. 21, 2000; Physician's Desk Reference (Thomson Publishing; and/or The Merck Manual of Diagnosis and Therapy, 18th ed., 2006, Beers and Berkow, Eds., Merck Publishing Group; or, in the case of animals, The Merck Veterinary Manual, 9th ed., Kahn Ed., Merck Publishing Group, 2005; all of which are incorporated herein by reference used herein to refer to an agent or a derivative thereof that can interact with a hyperproliferative cell such as a cancer cell or an immune cell, thereby reducing the proliferative status of the cell and/or killing the cell. Examples of therapeutic agents include, but are not limited to, chemotherapeutic agents, cytotoxic agents, platinum-based agents (e.g., cisplatin, carboplatin, oxaliplatin), taxanes (e.g., Taxol), etoposide, alkylating agents (e.g., cyclophosphamide, ifosamide), metabolic antagonists (e.g., methotrexate (MTX), 5-fluorouracil, gemcitabine, pemetrexed, or derivatives thereof), antitumor antibiotics (e.g., mitomycin, doxorubicin), plant-derived antitumor agents (e.g., vincristine, vindesine, Taxol). Such agents may further include, but are not limited to, the anticancer agents trimetrexate, TEMOZOLOMIDE™, RALTRITREXED™, S-(4-Nitrobenzyl)-6-thioinosine (NBMPR), 6-benzyguanidine (6-BG), bis-chloronitrosourea (BCNU) and CAMPTOTHECIN™, or a therapeutic derivative of any thereof. “Therapeutic agents” also refer to salts, acids, and free based forms of the above agents.

The term “kit” refers to a set of two or more components necessary for employing the methods and compositions provided herein. Kit components can include, but are not limited to, carotenoids, polyethylene glycol, and aqueous solutions, liposome compositions and pharmaceutical compositions disclosed herein, reagents, buffers, containers and/or equipment.

The term “radiosensitizing agent” means a compound that makes tumor cells more sensitive to radiation therapy. Examples of radiosensitizing agents include misonidazole, metronidazole, tirapazamine, and trans-crocetin.

### Aqueous Solutions and Pharmaceutical Compositions

The provided aqueous solutions and pharmaceutical compositions can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith et al., March's Advanced Organic Chemistry. Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

In some embodiments, the disclosure provides a solution comprising an ionizable carotenoid salt and low molecular weight polyethylene glycol.

In some embodiments, the disclosure provides an aqueous solution comprising an ionizable carotenoid salt having the formula:

Q-Polyene Carotenoid-Q,
wherein,
   the Polyene Carotenoid comprises
(a) 3, 4, 5, 6, 7, 8, 9, 10, 3-5, 6-8, 9-10, or more than 9, conjugated double bonds,
(b) methyl or low alkyl (C2-C3) substitutions, and
(c) 1, 2, 3, or more than 3, ionizable groups, and
Q is a monovalent counterion; and
polyethylene glycol having a molecular weight of 200-700 Da.

In some embodiments, the Polyene Carotenoid comprises all trans conjugated double bonds. In some embodiments, the Polyene Carotenoid comprises 6-9 conjugated double bonds. In particular embodiments, the Polyene Carotenoid comprises 7 conjugated double bonds. The Polyene Carotenoid can be naturally occurring or synthetic. In some embodiments, the Polyene Carotenoid is naturally occurring. In other embodiments, the Polyene Carotenoid is synthetic. The ionizable group(s) may be anionic and/or cationic. In some embodiments, the Polyene Carotenoid comprises two or more of the same ionizable group. In some embodiments, the Polyene Carotenoid comprises two or more different ionizable groups. In some embodiments, the Polyene Carotenoid comprises one or more anionic ionizable groups. In some embodiments, the Polyene Carotenoid comprises at least one ionizable group selected from: a carboxylic group, a sulfonate group, a sulfate group, a phosphonate, or a phosphate group, and a hydroxamate moiety. In other embodiments, the Polyene Carotenoid comprises one or more cationic ionizable groups (e.g., a primary, secondary, or tertiary amine group, a quaternary ammonium group, a choline group, a guanidine group, or an imidazole group). In particular embodiments, the Polyene Carotenoid comprises one or more cationic ionizable groups and the pharmaceutical composition is substantially free of nucleic acids.

In some embodiments, the ionizable carotenoid salt concentration of the Q-Polyene Carotenoid-Q aqueous solution or pharmaceutical composition is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, or at least 10 mg/mL.

In some embodiments, the ionizable carotenoid salt concentration of the Q-Polyene Carotenoid-Q aqueous solution or pharmaceutical composition is 0.01 mg/mL to 30 mg/mL, 0.05 mg/mL to 25 mg/mL, 0.075 mg/mL to 20 mg/mL, 0.1 mg/mL to 15 mg/mL, 0.5 mg/mL to 10 mg/mL, 1 mg/mL to 8 mg/mL, 1.5 mg/mL to 6 mg/mL, or 2 mg/mL to 5 mg/mL (e.g., 2 mg/mL, 3 mg/ml, 4 mg/mL, or 5 mg/mL). In particular embodiments, the ionizable carotenoid salt concentration of the aqueous solution is 2 mg/mL to 10 mg/mL. In particular embodiments, the ionizable carotenoid salt concentration of the aqueous solution is 2 mg/mL to 5 mg/mL. In further embodiments, the concentration of the ionizable carotenoid salt in the aqueous solution or pharmaceutical composition is about 2 mg/mL, about 3 mg/ml, about 4 mg/mL, about 5 mg/mL about 6 mg/mL about 7 mg/mL about 8 mg/mL, about 9 mg/mL, or about 10 mg/mL.

In some embodiments, Q is a monovalent metal cation. In some embodiments, Q is at least one member selected from NH₄⁺, Na⁺, Li⁺, K⁺ or a monovalent organic cation such as protonated amine. In some embodiments, Q is Na⁺. In further embodiments, the Q-Polyene Carotenoid-Q is sodium trans-crocetinate (STC). In some embodiments, Q is K⁺. In further embodiments, the Q-Polyene Carotenoid-Q is potassium trans-crocetinate (KTC).

In some embodiments, the PEG in the Q-Polyene Carotenoid-Q aqueous solution has an average molecular weight between 200-700 Da, 200-600 Da, 300-500 Da, or 350-450 Da, (e.g., 400 Da). In further embodiments, the PEG is PEG-200, PEG-300, PEG-400, PEG-500, or PEG-600. In particular embodiments, the Q-Polyene Carotenoid-Q aqueous solution comprises PEG-400.

In some embodiments, the concentration of PEG in the Q-Polyene Carotenoid-Q aqueous solution is 0.01% to 40%, 0.05% to 35%, 0.1% to 20%, 0.5% to 15%, 1% to 10%, 2% to 9%, 3% to 8%, or 5% to 7% (w/w). In further embodiments, the ionizable carotenoid salt to PEG ratio in the aqueous solution is 1:1-300; 1:1-100; 1-200; 1:1-50; 1:1-40, 1:5-30; or 1:10-25 (e.g., 1:20) (w/w).

In some embodiments, PEG in the Q-Polyene Carotenoid-Q aqueous solution has an average molecular weight between 200-600 Da and the ionizable carotenoid salt to PEG ratio is 1:1-300; 1:1-100; 1-200; 1:1-50; 1:1-40, 1:5-30; or 1:10-25 (e.g., 1:20) (w/w). In particular embodiments the PEG in the Q-Polyene Carotenoid-Q aqueous solution has an average molecular weight of about 400 Da and the ionizable carotenoid salt to PEG ratio is 1:1-300; 1:1-100; 1-200; 1:1-50; 1:1-40, 1:5-30; or 1:10-25 (e.g., 1:20). (w/w). In further particular embodiments the PEG in the Q-Polyene Carotenoid-Q aqueous solution has an average molecular weight of about 400 Da and the ionizable carotenoid salt to PEG ratio is 1:20.

In particular embodiments, the Q-Polyene Carotenoid-Q aqueous solution contains an ionizable carotenoid salt concentration of 0.01 mg/mL to 30 mg/mL, and PEG having an average molecular weight between 200-700 Da at a concentration of 0.05% to 35% (w/w). In further embodiments, the aqueous solution contains an ionizable carotenoid salt concentration of 0.1 mg/mL to 15 mg/mL, and PEG having an average molecular weight between 200-600 Da at a concentration of 1% to 10% (e.g., 2% to 7%) (w/w).

In some embodiments, the Q-Polyene Carotenoid-Q aqueous solution has a pH of 6-10, 7.5-9.5, 8-9 (e.g., pH 8.5), or any range therein between. In some embodiments, the aqueous solution comprises a buffer having a pKA within 1 unit or within 0.5 units of the pH of the solution at a concentration of 1-200 mM, 1-100 mM, 1-80 mM, or any range therein between. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate. In some embodiments, the aqueous solution comprises glycine. In some embodiments, the aqueous solution comprises gly-gly. In additional embodiments, the aqueous solution comprises sodium bicarbonate. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate at a concentration of 1-200 mM, 1-100 mM, 1-80 mM, or any range therein between. In some embodiments, the aqueous solution comprises glycine at a concentration of 1-200 mM, 1-100 mM, 1-80 mM, or any range therein between. In some embodiments, the aqueous solution comprises gly-gly. at a concentration of 1-200 mM, 1-100 mM, 1-80 mM, or any range therein between. In additional embodiments, the aqueous solution comprises sodium bicarbonate at a concentration of 1-200 mM, 1-100 mM, 1-80 mM, or any range therein between.

In further embodiments, the Q-Polyene Carotenoid-Q aqueous solution further comprises a tonicity controlling agent. In some embodiments, the aqueous solution comprises at least one tonicity controlling agent selected from sodium chloride, mannitol, sorbitol, xylitol, dextrose, maltose, glucose, lactose and sucrose. In particular embodiments, the aqueous solution comprises sucrose. In other particular embodiments, the aqueous solution comprises sodium chloride. In some embodiments, the Q-Polyene Carotenoid-Q aqueous solution contains a tonicity controlling agent (e.g., sucrose or NaCl) at a convention of 0.05 mM to 100 mM, 0.75 mM to 75 mM, 1 mM to 50 mM, 5 mM to 40 mM, 7 mM to 30 mM, or 10 mM to 20 mM.

Pharmaceutical compositions comprising the Q-Polyene-Carotenoid-Q aqueous solutions are also encompassed by the disclosure.

In some embodiments, the disclosure provides an aqueous solution comprising an ionizable carotenoid salt having the formula:

Q-R₁-Polyene Carotenoid-R₂-Q,
wherein,
   the Polyene Carotenoid comprises
(a) 3, 4, 5, 6, 7, 8, 9, 10, 3-5, 6-8, 9-10, or more than 9, conjugated double bonds,
(b) methyl or low alkyl (C2-C3) substitutions, and
(c) 1, 2, 3, or more than 3, ionizable groups;
R₁ and R₂ are ionizable groups (e.g., the same ionizable group or different ionizable groups), and
Q is a monovalent counterion; and
polyethylene glycol having a molecular weight of 200-700 Da.

In some embodiments, the R1-Polyene Carotenoid-R2 in the aqueous solution comprises all trans conjugated double bonds. In particular embodiments, the R1-Polyene Carotenoid-R2 comprises 6-9 conjugated double bonds. The R1-Polyene Carotenoid-R2 can be naturally occurring or synthetic. In some embodiments, the R1-Polyene Carotenoid-R2 is naturally occurring. In other embodiments, the R1-Polyene Carotenoid-R2 is synthetic. In some embodiments, R₁ and R₂ are the same ionizable group. In other embodiments, R₁ and R₂ are different ionizable groups. In some embodiments, R₁ and R₂ are the same cationic ionizable group (e.g., a primary, secondary, or tertiary amine group, a quaternary ammonium group, a choline group, a guanidine group, and an imidazole group). In other embodiments, R₁ and R₂ are different cationic groups. In some embodiments, R₁ and R₂ are the same anionic ionizable group (e.g., a carboxylic group, a sulfonate group, a sulfate group, a phosphonate, a phosphate group, and a hydroxamate group). In other embodiments, R₁ and R₂ are different anionic groups. In some embodiments, R₁ is a cationic ionizable group or anionic ionizable group and R₂ is an anionic ionizable group or cationic group, respectively. In some embodiments, the R1-Polyene Carotenoid-R2 comprises at least one ionizable group selected from: a carboxylic group, a sulfonate group, a sulfate group, a phosphonate, or a phosphate group, and a hydroxamate moiety. In some embodiments, R₁ and/or R₂ is at least one ionizable group selected from: a carboxylic group, a sulfonate group, a sulfate group, a phosphonate, or a phosphate group, and a hydroxamate moiety. In some embodiments, R₂ is at least one ionizable group selected from: a carboxylic group, a sulfonate group, a sulfate group, a phosphonate, or a phosphate group, and a hydroxamate moiety. In other embodiments, R₁ and/or R₂ a cationic ionizable group (e.g., a primary, secondary, or tertiary amine group, a quaternary ammonium group, a choline group, a guanidine group, or an imidazole group). In particular embodiments, R₁ is a cationic ionizable group and the pharmaceutical composition is substantially free of nucleic acids.

In some embodiments, the ionizable carotenoid salt concentration of the R1-Polyene Carotenoid-R2 aqueous solution is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, or at least 10 mg/mL.

In some embodiments, the ionizable carotenoid salt concentration of the Q-R1-Polyene Carotenoid-R2-Q aqueous solution is 0.01 mg/mL to 30 mg/mL, 0.05 mg/mL to 25 mg/mL, 0.075 mg/mL to 20 mg/mL, 0.1 mg/mL to 15 mg/mL, 0.5 mg/mL to 10 mg/mL, 1 mg/mL to 8 mg/mL, 1.5 mg/mL to 6 mg/mL, or 2 mg/mL to 5 mg/mL (e.g., 2 mg/mL, 3 mg/ml, 4 mg/mL, or 5 mg/mL). In particular embodiments, the ionizable carotenoid salt concentration of the aqueous solution is 2 mg/mL to 10 mg/mL. In particular embodiments, the ionizable carotenoid salt concentration of the aqueous solution is 2 mg/mL to 5 mg/mL. In further embodiments, the ionizable carotenoid salt concentration of the aqueous solution or pharmaceutical composition is about 2 mg/mL, about 3 mg/ml, about 4 mg/mL, about 5 mg/mL about 6 mg/mL about 7 mg/mL about 8 mg/mL. about 9 mg/mL, or about 10 mg/mL.

In some embodiments, Q is a monovalent metal cation. In some embodiments, Q is at least one member selected from NH₄⁺, Na⁺, Li⁺, K⁺ or a monovalent organic cation such as protonated amine. In some embodiments, Q is Na⁺. In further embodiments, the Q-R1-Polyene Carotenoid-R2-Q is sodium trans-crocetinate (STC). In some embodiments, Q is K⁺. In further embodiments, the Q-R1-Polyene Carotenoid-R2-Q is potassium trans-crocetinate (KTC).

In some embodiments, the PEG in the Q-R1-Polyene Carotenoid-R2-Q aqueous solution has an average molecular weight between 200-700 Da, 200-600 Da, 300-500 Da, or 350-450 Da, (e.g., 400 Da). In further embodiments, the PEG is PEG-200, PEG-300, PEG-400, PEG-500, or PEG-600. In particular embodiments, the Q-Polyene Carotenoid-Q aqueous solution comprises PEG-400.

In some embodiments, the concentration of PEG in the Q-R1-Polyene Carotenoid-R2-Q aqueous solution is 0.01% to 40%, 0.05% to 35%, 0.1% to 20%, 0.5% to 15%, 1% to 10%, 2% to 9%, 3% to 8%, or 5% to 7% (w/w). In further embodiments, the ionizable carotenoid salt to PEG ratio in the aqueous solution is 1:1-300; 1:1-100; 1-200; 1:1-50; 1:1-40, 1:5-30; or 1:10-25 (e.g., 1:20) (w/w).

In some embodiments, PEG in the Q-R1-Polyene Carotenoid-R2-Q aqueous solution has an average molecular weight between 200-600 Da and the ionizable carotenoid salt to PEG ratio is 1:1-300; 1:1-100; 1-200; 1:1-50; 1:1-40, 1:5-30; or 1:10-25 (e.g., 1:20) (w/w). In particular embodiments the PEG in the Q-R1-Polyene Carotenoid-R2-Q aqueous solution has an average molecular weight of about 400 Da and the ionizable carotenoid salt to PEG ratio is 1:1-300; 1:1-100; 1-200; 1:1-50; 1:1-40, 1:5-30; or 1:10-25 (e.g., 1:20). (w/w). In further particular embodiments the PEG in the Q-R1-Polyene Carotenoid-R2-Q aqueous solution has an average molecular weight of about 400 Da and the ionizable carotenoid salt to PEG ratio is 1:20.

In particular embodiments, the Q-R1-Polyene Carotenoid-R2-Q aqueous solution contains an ionizable carotenoid salt concentration of 0.01 mg/mL to 30 mg/mL, and PEG having an average molecular weight between 200-700 Da at a concentration of 0.05% to 35% (w/w). In further embodiments, the aqueous solution contains an ionizable carotenoid salt concentration of 0.1 mg/mL to 15 mg/mL, and PEG having an average molecular weight between 200-600 Da at a concentration of 1% to 10% (e.g., 2% to 7%) (w/w).

In some embodiments, the Q-R₁-Polyene Carotenoid-R₂-Q aqueous solution has a pH of 6-10, 7.5-9.5, 8-9 (e.g., pH 8.5), or any range therein between. In some embodiments, the aqueous solution comprises a buffer having a pKA within 1 unit or within 0.5 units of the pH of the solution at a concentration of 1-200 mM, 1-100 mM, 1-80 mM, or any range therein between. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate. In some embodiments, the aqueous solution comprises glycine. In some embodiments, the aqueous solution comprises gly-gly. In additional embodiments, the aqueous solution comprises sodium bicarbonate. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate at a concentration of 1-200 mM, 1-100 mM, 1-80 mM, or any range therein between. In some embodiments, the aqueous solution comprises glycine at a concentration of 1-200 mM, 1-100 mM, 1-80 mM, or any range therein between. In some embodiments, the aqueous solution comprises gly-gly. at a concentration of 1-200 mM, 1-100 mM, 1-80 mM, or any range therein between. In additional embodiments, the aqueous solution comprises sodium bicarbonate at a concentration of 1-200 mM, 1-100 mM, 1-80 mM, or any range therein between.

In further embodiments, the Q-R1-Polyene Carotenoid-R2-Q aqueous solution further comprises a tonicity controlling agent. In some embodiments, the aqueous solution comprises at least one tonicity controlling agent selected from sodium chloride, mannitol, sorbitol, xylitol, dextrose, maltose, glucose, lactose and sucrose. In particular embodiments, the aqueous solution comprises sucrose. In other particular embodiments, the aqueous solution comprises sodium chloride. In some embodiments, the Q-R1-Polyene Carotenoid-R2-Q aqueous solution contains a tonicity controlling agent (e.g., sucrose or NaCl) at a convention of 0.05 mM to 100 mM, 0.75 mM to 75 mM, 1 mM to 50 mM, 5 mM to 40 mM, 7 mM to 30 mM, or 10 mM to 20 mM.

Pharmaceutical compositions comprising the Q-R1-Polyene Carotenoid-R2-Q aqueous solutions are also encompassed by the disclosure.

In some embodiments, the disclosure provides an aqueous solution comprising an ionizable carotenoid salt having the formula:

Q-trans-crocetin-Q,
wherein,
Q is a monovalent cation counterion, and
polyethylene glycol having a molecular weight of 200-700 Da.

In some embodiments, the crocetin concentration of the aqueous solution is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, or at least 10 mg/mL.

In some embodiments, the trans-crocetin concentration of the aqueous solution is 0.01 mg/mL to 30 mg/mL, 0.05 mg/mL to 25 mg/mL, 0.075 mg/mL to 20 mg/mL, 0.1 mg/mL to 15 mg/mL, 0.5 mg/mL to 10 mg/mL, 1 mg/mL to 8 mg/mL, 1.5 mg/mL to 6 mg/mL, or 2 mg/mL to 5 mg/mL (e.g., 2 mg/mL, 3 mg/ml, 4 mg/mL, or 5 mg/mL). In particular embodiments, the crocetin concentration of the aqueous solution is 2 mg/mL to 10 mg/mL. In particular embodiments, the crocetin concentration of the aqueous solution is 2 mg/mL to 5 mg/mL. In further embodiments, the concentration of the crocetin in the aqueous solution or pharmaceutical composition is about 2 mg/mL, about 3 mg/ml, about 4 mg/mL, about 5 mg/mL about 6 mg/mL about 7 mg/mL about 8 mg/mL, about 9 mg/mL, or about 10 mg/mL.

In some embodiments, Q is a monovalent metal cation. In some embodiments, Q is at least one member selected from NH₄⁺, Na⁺, Li⁺, K⁺ or a monovalent organic cation such as protonated amine. In some embodiments, Q is Na⁺ and the Q-trans-crocetin-Q is sodium trans-crocetinate (STC). In some embodiments, Q is K⁺ and the Q-trans-crocetin-Q is potassium trans-crocetinate (KTC).

In some embodiments, the PEG in the Q-trans-crocetin-Q aqueous solution has an average molecular weight between 200-700 Da, 200-600 Da, 300-500 Da, or 350-450 Da, (e.g., 400 Da). In further embodiments, the PEG is PEG-200, PEG-300, PEG-400, PEG-500, or PEG-600. In particular embodiments, the Q-trans-crocetin-Q aqueous solution comprises PEG-400.

In some embodiments, the concentration of PEG in the Q-trans-crocetin-Q aqueous solution is 0.01% to 40%, 0.05% to 35%, 0.1% to 20%, 0.5% to 15%, 1% to 10%, 2% to 9%, 3% to 8%, or 5% to 7% (w/w). In further embodiments, the trans-crocetin salt to PEG ratio in the aqueous solution is 1:1-300; 1:1-100; 1-200; 1:1-50; 1:1-40, 1:5-30; or 1:10-25 (e.g., 1:20) (w/w).

In some embodiments, PEG in the Q-trans-crocetin-Q aqueous solution has an average molecular weight between 200-600 Da and the trans-crocetin salt to PEG ratio is 1:1-300; 1:1-100; 1-200; 1:1-50; 1:1-40, 1:5-30; or 1:10-25 (e.g., 1:20) (w/w). In particular embodiments the PEG in the Q-trans-crocetin-Q aqueous solution has an average molecular weight of about 400 Da and the ionizable carotenoid salt to PEG ratio is 1:1-300; 1:1-100; 1-200; 1:1-50; 1:1-40, 1:5-30; or 1:10-25 (e.g., 1:20). (w/w). In further particular embodiments the PEG in the Q-trans-crocetin-Q aqueous solution has an average molecular weight of about 400 Da and the ionizable carotenoid salt to PEG ratio is 1:20.

In particular embodiments, the Q-trans-crocetin-Q aqueous solution contains a trans-crocetin concentration of 0.01 mg/mL to 30 mg/mL, and PEG having an average molecular weight between 200-700 Da at a concentration of 0.05% to 35% (w/w). In further embodiments, the aqueous solution contains a trans-crocetin concentration of 0.1 mg/mL to 15 mg/mL, and PEG having an average molecular weight between 200-600 Da at a concentration of 1% to 10% (e.g., 2% to 7%) (w/w).

In some embodiments, the Q-R₁-Polyene Carotenoid-R₂-Q aqueous solution has a pH of 6-10, 7.5-9.5, 8-9 (e.g., pH 8.5), or any range therein between. In some embodiments, the aqueous solution comprises a buffer having a pKA within 1 unit or within 0.5 units of the pH of the solution at a concentration of 1-200 mM, 1-100 mM, 1-80 mM, or any range therein between. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate. In some embodiments, the aqueous solution comprises glycine. In some embodiments, the aqueous solution comprises gly-gly. In additional embodiments, the aqueous solution comprises sodium bicarbonate. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate at a concentration of 1-200 mM, 1-100 mM, 1-80 mM, or any range therein between. In some embodiments, the aqueous solution comprises glycine at a concentration of 1-200 mM, 1-100 mM, 1-80 mM, or any range therein between. In some embodiments, the aqueous solution comprises gly-gly. at a concentration of 1-200 mM, 1-100 mM, 1-80 mM, or any range therein between. In additional embodiments, the aqueous solution comprises sodium bicarbonate at a concentration of 1-200 mM, 1-100 mM, 1-80 mM, or any range therein between.

In some embodiments, the provided aqueous solution has a pH of 6-10, or any range therein between. In some embodiments, the aqueous solution has a pH of 7.5-9.5, or any range therein between. In some embodiments, the aqueous solution has a pH of 8-9 (e.g., pH 8.5), or any range therein between. In some embodiments, the aqueous solution has a pH of 6-10, 7.5-9.5, or 8-9 (e.g., pH 8.5), or any range therein between.

In additional embodiments, the provided aqueous composition comprises a buffer. In some embodiments, the aqueous solution comprises a buffer having a pKA within 1 unit or within 0.5 units of the pH of the solution at a concentration of 1-200 mM, 1-100 mM, 1-80 mM, or any range therein between.

In further embodiments, the buffer is selected from glycine, gly-gly, sodium bicarbonate, sodium phosphate, tricine, bicine, EPPS (HEPPS), HEPBS, TABS, AMPD, or sodium borate. In a particular embodiment, the buffer is glycine. In another embodiment, the buffer is gly-gly. In a further embodiment, the buffer is sodium bicarbonate. In some embodiments, the buffer is at a concentration of 1-200 mM, 1-100 mM, 1-80 mM, or any range therein between. In some embodiments, the buffer is at a concentration of 1 to 200 mM, or any range therein between. In yet further embodiments, the buffer is at a concentration of 1-100 mM, or any range therein between. In yet further embodiments, the buffer is at a concentration of 1-80 mM, or any range therein between. In yet further embodiments, the buffer is at a concentration of 1-50 mM, or any range therein between. In yet further embodiments, the buffer is at a concentration of 1-25 mM, or any range therein between.

In further embodiments, the trans-crocetin aqueous solution further comprises a tonicity controlling agent. In some embodiments, the aqueous solution comprises at least one tonicity controlling agent selected from sodium chloride, mannitol, sorbitol, xylitol, dextrose, maltose, glucose, lactose and sucrose. In particular embodiments, the aqueous solution comprises sucrose. In other particular embodiments, the aqueous solution comprises sodium chloride. In some embodiments, the trans-crocetin aqueous solution contains a tonicity controlling agent (e.g., sucrose or NaCl) at a concentration of 0.05 mM to 100 mM, 0.75 mM to 75 mM, 1 mM to 50 mM, 5 mM to 40 mM, 7 mM to 30 mM, or 10 mM to 20 mM.

Pharmaceutical compositions comprising the trans-crocetin aqueous solutions are also encompassed by the disclosure.

In some embodiments, the disclosure provides an aqueous solution comprising an ionizable carotenoid salt having the formula:

Q-trans-norbixin-Q,
wherein,
Q is a monovalent cation counterion, and
polyethylene glycol having a molecular weight of 200-700 Da.

In some embodiments, the norbixin concentration of the aqueous solution is at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, or at least 10 mg/mL.

In some embodiments, the trans-norbixin concentration of the aqueous solution is 0.01 mg/mL to 30 mg/mL, 0.05 mg/mL to 25 mg/mL, 0.075 mg/mL to 20 mg/mL, 0.1 mg/mL to 15 mg/mL, 0.5 mg/mL to 10 mg/mL, 1 mg/mL to 8 mg/mL, 1.5 mg/mL to 6 mg/mL, or 2 mg/mL to 5 mg/mL (e.g., 2 mg/mL, 3 mg/ml, 4 mg/mL, or 5 mg/mL). In particular embodiments, the norbixin concentration of the aqueous solution is 2 mg/mL to 10 mg/mL. In particular embodiments, the norbixin concentration of the aqueous solution is 2 mg/mL to 5 mg/mL. In further embodiments, the concentration of the norbixin in the aqueous solution or pharmaceutical composition is about 2 mg/mL, about 3 mg/ml, about 4 mg/mL, about 5 mg/mL about 6 mg/mL about 7 mg/mL about 8 mg/mL, about 9 mg/mL, or about 10 mg/mL.

In some embodiments, Q is a monovalent metal cation. In some embodiments, Q is at least one member selected from NH₄⁺, Na⁺, Li⁺, K⁺ or a monovalent organic cation such as protonated amine. In some embodiments, Q is Na⁺ and the Q-trans-norbixin-Q is sodium trans-norbixinate (STC). In some embodiments, Q is K⁺ and the Q-trans-norbixin-Q is potassium trans-norbixinate (KTC).

In some embodiments, the PEG in the Q-trans-norbixin-Q aqueous solution has an average molecular weight between 200-700 Da, 200-600 Da, 300-500 Da, or 350-450 Da, (e.g., 400 Da). In further embodiments, the PEG is PEG-200, PEG-300, PEG-400, PEG-500, or PEG-600. In particular embodiments, the Q-trans-norbixin-Q aqueous solution comprises PEG-400.

In some embodiments, the concentration of PEG in the Q-trans-norbixin-Q aqueous solution is 0.01% to 40%, 0.05% to 35%, 0.1% to 20%, 0.5% to 15%, 1% to 10%, 2% to 9%, 3% to 8%, or 5% to 7% (w/w). In further embodiments, the trans-norbixin salt to PEG ratio in the aqueous solution is 1:1-300; 1:1-100; 1-200; 1:1-50; 1:1-40, 1:5-30; or 1:10-25 (e.g., 1:20) (w/w).

In some embodiments, PEG in the Q-trans-norbixin-Q aqueous solution has an average molecular weight between 200-600 Da and the trans-norbixin salt to PEG ratio is 1:1-300; 1:1-100; 1-200; 1:1-50; 1:1-40, 1:5-30; or 1:10-25 (e.g., 1:20) (w/w). In particular embodiments the PEG in the Q-trans-norbixin-Q aqueous solution has an average molecular weight of about 400 Da and the ionizable carotenoid salt to PEG ratio is 1:1-300; 1:1-100; 1-200; 1:1-50; 1:1-40, 1:5-30; or 1:10-25 (e.g., 1:20). (w/w). In further particular embodiments the PEG in the Q-trans-norbixin-Q aqueous solution has an average molecular weight of about 400 Da and the ionizable carotenoid salt to PEG ratio is 1:20.

In particular embodiments, the Q-trans-norbixin-Q aqueous solution contains a trans-norbixin concentration of 0.01 mg/mL to 30 mg/mL, and PEG having an average molecular weight between 200-700 Da at a concentration of 0.05% to 35% (w/w). In further embodiments, the aqueous solution contains a trans-norbixin concentration of 0.1 mg/mL to 15 mg/mL, and PEG having an average molecular weight between 200-600 Da at a concentration of 1% to 10% (e.g., 2% to 7%) (w/w).

In some embodiments, the Q-trans-norbixin-Q aqueous solution has a pH of 6-10, 7.5-9.5, 8-9 (e.g., pH 8.5), or any range therein between. In some embodiments, the aqueous solution comprises a buffer having a pKA within 1 unit or within 0.5 units of the pH of the solution at a concentration of 1-200 mM, 1-100 mM, 1-80 mM, or any range therein between. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate. In some embodiments, the aqueous solution comprises glycine. In some embodiments, the aqueous solution comprises gly-gly. In additional embodiments, the aqueous solution comprises sodium bicarbonate. In some embodiments, the aqueous solution comprises a buffer selected from: glycine, gly-gly, sodium bicarbonate, sodium phosphate, tricine, bicine, EPPS (HEPPS), HEPBS, TABS, AMPD, or sodium borate at a concentration of 1-200 mM, 1-100 mM, 1-80 mM, or any range therein between. In some embodiments, the aqueous solution comprises glycine at a concentration of 1-200 mM, 1-100 mM, 1-80 mM, or any range therein between. In some embodiments, the aqueous solution comprises gly-gly. at a concentration of 1-200 mM, 1-100 mM, 1-80 mM, or any range therein between. In additional embodiments, the aqueous solution comprises sodium bicarbonate at a concentration of 1-200 mM, 1-100 mM, 1-80 mM, or any range therein between.

In further embodiments, the trans-norbixin aqueous solution further comprises a tonicity controlling agent. In some embodiments, the aqueous solution comprises at least one tonicity controlling agent selected from sodium chloride, mannitol, sorbitol, xylitol, dextrose, maltose, glucose, lactose and sucrose. In particular embodiments, the aqueous solution comprises sucrose. In other particular embodiments, the aqueous solution comprises sodium chloride. In some embodiments, the trans-norbixin aqueous solution contains a tonicity controlling agent (e.g., sucrose or NaCl) at a concentration of 0.05 mM to 100 mM, 0.75 mM to 75 mM, 1 mM to 50 mM, 5 mM to 40 mM, 7 mM to 30 mM, or 10 mM to 20 mM.

Pharmaceutical compositions comprising the trans-norbixin aqueous solutions are also encompassed by the disclosure.

### Formulation and Administration

The provided compositions can be formulated in whole or in part as pharmaceutical compositions. Pharmaceutical compositions may include one or more nanoparticle compositions. For example, a pharmaceutical composition may include one or more nanoparticle compositions including one or more different therapeutic and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

In some embodiments, the disclosure provides an aqueous solution or pharmaceutical composition and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions provided herein (See, e.g., Remington's Pharmaceutical Sciences, 17th ed., 1989).

The provided compositions may be sterilized by conventional, known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions. In some embodiments, the pharmaceutical composition comprises a tonicity agent at a concentration of greater than 0.1%, or a concentration of 0.3% to 2.5%, 0.5% to 2.0%, 0.5% to 1.5%, 0.5% to 1.5%, 0.6% to 1.1%, or any range therein between. In some embodiments, the pharmaceutical composition comprises a tonicity agent such as dextrose, mannitol, glycerin, potassium chloride, or sodium chloride. In further embodiments, the pharmaceutical composition comprises dextrose, mannitol, glycerin, potassium chloride, or sodium chloride at a concentration of greater than 0.1%, or a concentration of 0.3% to 2.5%, 0.5% to 2.0%, 0.5% to 1.5%, 0.5% to 1.5%, 0.6% to 1.1%, or any range therein between.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In some embodiments, the provided pharmaceutical compositions are administered, for example, by intravenous infusion, topically, intraperitoneally, intravesically, or intrathecally. In particular embodiments, the pharmaceutical compositions are parentally or intravenously administered. Preferably, the pharmaceutical compositions are administered parentally, i.e. intraarticularly, intravenously, subcutaneously, or intramuscularly. In other embodiments, the pharmaceutical preparation may be administered topically.

In some embodiments, the provided pharmaceutical compositions are presented in unit-dose or multi-dose sealed containers, such as ampoules and vials.

In some embodiments, the pharmaceutical preparations are administered in unit dosage form. In such form the preparation is subdivided into unit doses containing appropriate quantities of the active component, e.g., a liposome composition and aqueous solution. The unit dosage form can be a packaged preparation, the package containing discrete quantities of preparation. The composition can, if desired, also contain other compatible therapeutic agents (e.g., as described herein).

In some embodiments, the pharmaceutical compositions provided herein can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the pharmaceutical composition being employed. For example, dosages can be empirically determined considering the type and stage of the disease, disorder or condition diagnosed in a particular patient. The dose administered to a patient, in the context of the provided pharmaceutical compositions (e.g., liposome compositions) should be sufficient to affect a beneficial therapeutic response in the patient over time. The size of the dose will also be determined by the existence, nature, and extent of any adverse side-effects that accompany the administration of a particular pharmaceutical composition in a particular patient. Determination of the proper dosage for a particular situation is within the skill of the practitioner. Generally, treatment is initiated with smaller dosages which are less than the optimum dose of the pharmaceutical composition. Thereafter, the dosage is increased by small increments until the optimum effect under circumstances is reached. For convenience, the total daily dosage may be divided and administered in portions during the day, if desired.

In particular embodiments, the administered pharmaceutical composition comprises trans-crocetin and is administered to a subject (e.g., human) at a dosage sufficient to achieve a trans-crocetin serum concentrion of about 0.4 to ug/ml to 49.2 ug/ml or any range therein between.

In particular embodiments, the trans-crocetin pharmaceutical composition is administered to a subject (e.g., human) at a dosage of about 0.05 mg/kg to about 5.5 mg/kg. In some embodiments the trans-crocetin pharmaceutical composition is administered at a dosage of about 0.75 mg/kg to about 5 mg/kg or about 0.1 mg/kg to about 2.5 mg/kg as an iv bolus until recovery (e.g., an increased PaO2/FiO2 ratio by at 5%-75%, or any range therein between). In some embodiments, the trans-crocetin pharmaceutical composition is administered at a dosage of about about 0.75 mg/kg to about 5.5 mg/kg, or about 1 mg/kg to about 5 mg/kg as a continuous infusion over 24 hours. In particular embodiments, the trans-crocetin pharmaceutical composition is administered to a subject (e.g., a human) experiencing acute lung distress (e.g., presenting symptoms such as having difficulty breathing, tachypnea, mental confusion due to low oxygen levels) and/or having a PaO2/FiO2 ratio of less than 300 mm Hg. In other particular embodiments, the trans-crocetin pharmaceutical composition is administered to a subject (e.g., a human) experiencing ARDS and/or having a PaO2/FiO2 ratio of less than 200 mm Hg. In other particular embodiments, the trans-crocetin pharmaceutical composition is administered to a subject in order to increase the patients PaO2/FiO2 ratio. In some embodiments, administration of the trans-crocetin pharmaceutical composition increases the patient's PaO2/FiO2 ratio by at 5%-75%, or any range therein between. In further embodiments, the administration of the trans-crocetin pharmaceutical composition increases the patient's PaO2/FiO2 ratio by at least 5%, 10%, 15%, 20%, 25%, 30% 40% or 50%.

In particular embodiments, the trans-crocetin pharmaceutical composition is administered to a subject (e.g., human) at a dosage of about 0.20 to about 0.3 mg/kg (e.g., 2.5 mg/kg), or any range therein between, every 2-6 hours (e.g., every 2 hours or every three hours) as an IV bolus until recovery (e.g., an increased PaO2/FiO2 ratio by at 5%-75%, or any range therein between). In particular embodiments, the trans-crocetin pharmaceutical composition is administered to a subject (e.g., a human) experiencing acute lung distress (e.g., presenting symptoms such as having difficulty breathing, tachypnea, mental confusion due to low oxygen levels) and/or having a PaO2/FiO2 ratio of less than 300 mm Hg. In other particular embodiments, the trans-crocetin pharmaceutical composition is administered to a subject (e.g., a human) experiencing ARDS and/or having a PaO2/FiO2 ratio of less than 200 mm Hg. In other particular embodiments, the trans-crocetin pharmaceutical composition is administered to a subject in order to increase the patients PaO2/FiO2 ratio. In some embodiments, administration of the trans-crocetin pharmaceutical composition increases the patient's PaO2/FiO2 ratio by at 5%-75%, or any range therein between. In further embodiments, the administration of the trans-crocetin pharmaceutical composition increases the patient's PaO2/FiO2 ratio by at least 5%, 10%, 15%, 20%, 25%, 30% 40% or 50%.

### Liposome Loading

In additional embodiments, the disclosure provides an active loading method to generate an ionizable carotenoid salt inside a liposome formulation using a metal salt or pH gradient.

In some embodiments, the disclosure provides a method of preparing a loaded liposomal formulation comprising an ionizable carotenoid salt inside a liposome using a soluble metal salt gradient, wherein the method comprises:
(a) mixing the aqueous solution according to any one of [1]-[45], [98], [99], [106], or [107] with a liposomal solution containing liposomes in a weak acid salt of a multivalent metal; and
(b) maintaining the mixture of (a) for a sufficient time to load carotenoid into liposomes.

Loading of ionizable carotenoid may be established by maintaining the ionizable carotenoid in the liposomal solution for a suitable amount of time at a suitable temperature. Depending on the composition of the liposome, and the temperature, pH, and chemical nature of the ionizable carotenoid, loading of the ionizable carotenoid may occur over a time period of minutes or hours. In some embodiments, loading is carried out at temperatures of, for example, 0° C. to 95° C., or 20° C. to 75° C., or any range therein, preferably from about 40° C. to about 80° C.

In some embodiments, the ionizable carotenoid is an ionizable carotenoid in any of the aqueous solutions of [1]-[45] (e.g., trans-crocetin and trans-norbixin). In some embodiments, the carotenoid is a carotenoid disclosed in any of FIG. 1A-FIG. 1D. In some embodiments, the weak acid is selected from acetic acid, gluconic acid, tartaric acid, glutamic acid, citric acid, formic acid, and glycinic acid. In some embodiments, the weak acid salt of a multivalent metal is used at a concentration from 0 mM to 2000 mM, or 50 mM to 500 mM, or any range therein between. In some embodiments, the multivalent metal is selected from Ca²⁺, Mg²⁺, Zn²⁺, Cu²⁺, Co²⁺, Fe²⁺ and Fe³⁺. In some embodiments, the weak acid is acetic acid and the multivalent metal is Ca²⁺ (i.e., the weak acid salt of the multivalent metal is calcium acetate). In some embodiments, the weak acid is acetic acid and the multivalent metal is Mg²⁺ (i.e., the weak acid salt of the multivalent metal is magnesium acetate). Pharmaceutical compositions prepared according to the provided methods are also encompassed by the disclosure. The liposomal solution is preferably a buffered solution. However, it is appreciated that any suitable solvent may be use to prepare and use the provided compositions. A preferred liposome solution has a pH at about physiological pH and comprises a buffer which has a buffering range to include physiological pH. A non-limiting example of a suitable buffer for the liposome solution is HEPES (e.g., 5 mM HEPES buffered saline pH 6.5). Pharmaceutical compositions prepared according to the method are also encompassed by the disclosure.

In some embodiments, the liposome loading method further comprises the step of heating and cooling the drug loading mixture.

In some embodiments, the disclosure further provides the step of (c) removing unencapsulated ionizable carotenoid from the liposome preparation prepared according to (b). In some embodiment, the removal is carried out by passing the liposome preparation through a gel filtration column equilibrated with a second aqueous buffered solution, centrifugation, or dialysis, or related techniques. After removal of unencapsulated ionizable carotenoid, the extent of ionizable carotenoid loading may be determined by measurement of ionizable carotenoid and lipid levels according to conventional techniques. Lipid and drug concentrations may be determined using any suitable method known in the art, such as scintillation counting, spectrophotometric assays, and high performance liquid chromatography. Replacement of the liposome preparation solution to remove unencapsulated carotenoid and counterion, such as sodium acetate, can be accomplished using any of various techniques, known in the art, including but not limited to chromatography of the liposome preparation through an extensive gel filtration column equilibrated with a second aqueous buffered solution, by centrifugation, extensive or repeated dialysis, exchange of the liposomal preparation, treating the liposomal preparation with chelating agents or by related techniques. Pharmaceutical compositions prepared according to the provided methods are also encompassed by the disclosure.

In some embodiments, the weak acid salt used in the loading method is an organic acid (e.g., an organic acid selected from acetic acid, gluconic acid, tartaric acid, glutamic acid, citric acid, formic acid, and glycinic acid).

In some embodiments, the multivalent metal used in the loading method is a bivalent metal (e.g., a bivalent metal selected from Ca²⁺, Mg²⁺, Zn²⁺, Cu²⁺, Co²⁺, and Fe²⁺), or a trivalent metal such as Fe³⁺.

In further embodiments, the disclosure provides a method of preparing a loaded liposomal composition comprising an ionizable carotenoid salt contained in the aqueous solutions of any one of [1]-[45], [98], [99], [106], or [107] carotenoid using a soluble acetate metal salt gradient (calcium acetate or magnesium acetate). In yet further embodiments, the loading method uses acetic acid as the weak acid and Ca²⁺ or Mg²⁺ as the bivalent metal is (i.e., the weak acid salt of the bivalent metal is calcium acetate or magnesium acetate, respectively).

In some embodiments, the disclosure further provides the step of (c) removing unencapsulated ionizable carotenoid from the liposome preparation prepared according to (b). In some embodiment, the removal is carried out by passing the liposome preparation through a gel filtration column equilibrated with a second aqueous buffered solution, centrifugation, or dialysis, or related techniques. After removal of unencapsulated ionizable carotenoid, the extent of ionizable carotenoid loading may be determined by measurement of ionizable carotenoid and lipid levels according to conventional techniques. Lipid and drug concentrations may be determined using any suitable method known in the art, such as scintillation counting, spectrophotometric assays, and high performance liquid chromatography. Replacement of the liposome preparation solution to remove unencapsulated carotenoid and counterion, such as sodium acetate, can be accomplished using any of various techniques, known in the art, including but not limited to chromatography of the liposome preparation through an extensive gel filtration column equilibrated with a second aqueous buffered solution, by centrifugation, extensive or repeated dialysis, exchange of the liposomal preparation, treating the liposomal preparation with chelating agents or by related techniques. Pharmaceutical compositions prepared according to the provided methods are also encompassed by the disclosure.

In some embodiments, the disclosure provides a method of preparing a liposomal composition containing trans-crocetin inside a liposome using a soluble metal salt gradient, wherein the method comprises:
(a) mixing the aqueous solution according to [39], or [41]-[45] with a liposomal solution containing liposomes in a weak acid salt of a multivalent metal; and
(b) maintaining the mixture of (a) for a sufficient time to load trans-crocetin into liposomes.

Loading of trans-crocetin may be established by maintaining the trans-crocetin in the liposomal solution for a suitable amount of time at a suitable temperature. Depending on the composition of the liposome, and the temperature, pH, and chemical nature of trans-crocetin, loading of the trans-crocetin may occur over a time period of minutes or hours. In some embodiments, loading is carried out at temperatures of, for example, 0° C. to 95° C., or 20° C. to 75° C., or any range therein, preferably from about 40° C. to about 80° C.

In some embodiments, the weak acid is selected from acetic acid, gluconic acid, tartaric acid, glutamic acid, citric acid, formic acid, and glycinic acid. In some embodiments, the weak acid salt of a multivalent metal is used at a concentration from 0 mM to 2000 mM, or 50 mM to 500 mM, or any range therein between. In some embodiments, the multivalent metal is selected from Ca²⁺, Mg²⁺, Zn²⁺, Cu²⁺, Co²⁺, Fe²⁺, and Fe³⁺. In some embodiments, the weak acid is acetic acid and the multivalent metal is Ca²⁺ (i.e., the weak acid salt of the multivalent metal is calcium acetate). In some embodiments, the weak acid is acetic acid and the multivalent metal is Mg²⁺ (i.e., the weak acid salt of the multivalent metal is magnesium acetate). Pharmaceutical compositions prepared according to the method are also encompassed by the disclosure. The liposomal solution is preferably a buffered solution. However, it is appreciated that any suitable solvent may be utilized to practice the provided compositions and methods. A preferred liposome solution has a pH at about physiological pH and comprises a buffer which has a buffering range to include physiological pH. Non-limiting example of suitable buffers for the liposome solution is 5 mM HEPES buffered saline pH 6.5. Pharmaceutical compositions prepared according to the method are also encompassed by the disclosure.

In some embodiments, the disclosure further provides the step of (c) removing unencapsulated trans-crocetin from the liposome preparation prepared according to (b). In some embodiment, the removal is carried out by passing the liposome preparation through a gel filtration column equilibrated with a second aqueous buffered solution, or by centrifugation, dialysis, or related techniques. After removal of unencapsulated trans-crocetin, the extent of trans-crocetin loading may be determined by measurement of trans-crocetin and lipid levels according to conventional techniques. Lipid and drug concentrations may be determined by employing any suitable method known in the art, such as scintillation counting, spectrophotometric assays, and high performance liquid chromatography. Replacement of the liposome preparation solution to remove unencapsulated trans-crocetin and counterion, such as sodium acetate, can be accomplished using any of various techniques, known in the art, including but not limited to chromatography of the liposome preparation through an extensive gel filtration column equilibrated with a second aqueous buffered solution, centrifugation, extensive or repeated dialysis, exchange of the liposomal preparation, treating the liposomal preparation with chelating agents or by related techniques. Pharmaceutical compositions prepared according to the provided methods are also encompassed by the disclosure.

Multivalent counterions used in accordance with the present disclosure can be encapsulated in liposomes according to techniques described herein or otherwise known in the art. These methods include, for example, passive encapsulation techniques described herein or otherwise known in the art. Loading of an ionizable carotenoid such as trans-crocetin may be established by maintaining the ionizable carotenoid in the liposomal solution for a suitable amount of time at a suitable temperature. Depending on the composition of the liposome, and the temperature, pH, and chemical nature of the ionizable carotenoid, loading of the ionizable carotenoid may occur over a time period of minutes or hours. In some embodiments, loading is carried out at temperatures of, for example, 0° C. to 95° C., or 20° C. to 75° C., or any range therein between, preferably from about 40° C. to about 80° C., or any range therein between.

The compositions and characteristics of the liposomes that can be loaded according to the provided methods is not particularly limited. The properties of liposomes are influenced by the nature of lipids used to make the liposomes. A wide variety of lipids have been used to make liposomes. These include cationic, anionic and neutral lipids. The liposomes loaded according to the provided methods may contain functionalized and/or non-functionalized lipids. In some embodiments, the liposomes comprising the carotenoid compositions (e.g., CTC and MTC) are anionic or neutral. In other embodiments, the provided liposomes are cationic. The determination of the charge (e.g., anionic, neutral or cationic) can routinely be determined by measuring the zeta potential of the liposome. The zeta potential of the liposome can be positive, zero or negative. In some embodiments, the zeta potential of the liposome is −150 to 150 mV, or −50 to 50 mV, or any range therein between. In some embodiments, the zeta potential of the liposome is less than or equal to zero. In some embodiments, the zeta potential of the liposome is −150 to 0, −50 to 0 mV, −40 to 0 mV, −30 to 0 mV, −25 to 0 mV, −20 to 0 mV, −10 to 0 mV, −9 to 0 mV, −8 to 0 mV, −7 to 0 mV, −6 to 0 mV, −5 to 0 mV, −4 to 0 mV, −3 to 0 mV, −2 to 0 mV, −1 to 0 mV, or −8 to 2 mV, or any range therein between. In other embodiments, the zeta potential of the liposome is more than zero. In some embodiments, the liposome has a zeta potential that is 0.2 to 150 mV, 1 to 50 mV, 1 to 40 mV, 1 to 30 mV, 1 to 25 mV, 1 to 20 mV, 1 to 15 mV, 1 to 10 mV, 1 to 5 mV, 2 to 10 mV, 3 to 10 mV, 4 to 10 mV, or 5 to 10 mV, or any range therein between.

In some embodiments, the liposomes loaded according the the disclosed methods include a steric stabilizer that increases their longevity in circulation. One or more steric stabilizers such as a hydrophilic polymer (polyethylene glycol (PEG)), a glycolipid (monosialo-ganglioside (GM1)) or others occupies the space immediately adjacent to the liposome surface and excludes other macromolecules from this space. Consequently, access and binding of blood plasma opsonins to the liposome surface are hindered, and thus interactions of macrophages with such liposomes, or any other clearing mechanism, are inhibited and longevity of the liposome in circulation is enhanced. In some embodiments, the steric stabilizer or the population of steric stabilizers is a PEG or a combination comprising PEG. In further embodiments, the steric stabilizer is a PEG or a combination comprising PEG with a number average molecular weight (Mn) of 200 to 5000 Daltons. These PEG(s) can be of any structure such as linear, branched, star or comb structure and are commercially available.

In some embodiments, the diameter of the liposomes loaded according the the disclosed methods have a mean diameter of for example, 20 nm to 500 nm (nanometer), or 20 nm to 200 nm, or any range therein between. In some embodiments, the liposomes have a mean diameter of 80 nm to 120 nm, or any range therein between.

Lipoosomal formulations and pharmaceutical compositions comprising loaded liposomes encapsulating an ionizable carotenoid salt prepared according to the provided loading methods are also encompassed by the disclosure. In some embodiments, the ionizable carotenoid is an ionizable carotenoid in any of compositions [1]-[45] (e.g., trans-crocetin and trans-norbixin). In some embodiments, the ionizable carotenoid is a carotenoid disclosed in any of FIG. 1A-FIG. 1D. In some embodiments, the disclosure provides a pharmaceutical composition comprising a liposome encapsulating an ionizable carotenoid, wherein the ionizable carotenoid is loaded into liposomes in the presence of intra-liposomal multivalent counterions (e.g., Ca²⁺, Mg²⁺, Zn²⁺, Cu²⁺, Co²⁺, and Fe²⁺, and Fe³⁺). In some embodiments, the multivalent counterions comprise Ca²⁺. In some embodiments, the multivalent counterions comprise Mg²⁺. In some embodiments, the multivalent counterions comprise Fe³⁺.

In some embodiments, the disclosure provides a liposomal formulations and pharmaceutical compositions comprising loaded liposomes encapsulating trans-crocetin that are prepared according to the provided loading methods In further embodiments, the liposomes were prepared by loading trans-crocetin in the presence of intra-liposomal multivalent counterions (e.g., Ca²⁺, Mg²⁺, Zn²⁺, Cu²⁺, Co²⁺, and Fe²⁺, and Fe³⁺). In some embodiments, the multivalent counterions comprise Ca²⁺. In some embodiments, the multivalent counterions comprise Mg²⁺. In some embodiments, the multivalent counterions comprise Fe³⁺.

### Methods of Treatment and Use

In additional embodiments, the disclosure provides a method for increasing the delivery of oxygen in a subject who has or is at risk for developing ischemia (e.g., tissue hypoperfusion), that comprises administering to the subject an effective amount of a pharmaceutical composition provided herein, such as a pharmaceutical composition of any one of [46], [47], [100] or [108], thereby increasing the delivery of oxygen to the tissues and/or organs in the subject. In some embodiments, the subject has or is at risk for developing ischemia. In some embodiments, the pharmaceutical composition is administered to the subject before, during or following surgery (e.g., transplantation; reattachment of severed extremities, body parts or soft tissues; graft surgery, and vascular surgery). In some embodiments, the pharmaceutical composition is administered to a subject who has or is at risk for developing a wound, a burn injury, an electrical injury, or exposure to ionizing radiation. In some embodiments, the pharmaceutical composition is administered to a subject who has or is at risk for developing peripheral vascular disease, coronary artery disease, stroke, thrombosis, a clot, chronic vascular obstruction or vasculopathy (e.g., secondary to diabetes, hypertension, or peripheral vascular disease), or cerebral ischemia, pulmonary hypertension (adult or neonate); sickle cell disease; neointimal hyperplasia or restenosis (following angioplasty or stenting). In some embodiments, the pharmaceutical composition is administered to a subject who has or is at risk for developing a myopathy, kidney disease; asthma or adult respiratory distress syndrome; Alzheimer's and other dementias secondary to compromised cranial blood flow. In some embodiments, the method comprises administering the pharmaceutical composition of any one of [46], [47], [100] or [108]) to the subject. Use of a pharmaceutical composition provided herein (e.g., the pharmaceutical composition of any one of [46], [47], [100] or [108]), in the manufacture of a medicament for increasing the delivery of oxygen in a subject is also provided herein. As are, pharmaceutical compositions of any one of [46], [47], [100] or [108] for use in a medical medicament.

Methods are also disclosed herein for increasing the delivery of oxygen in a neonate subject or a subject who is elderly that comprises administering to the subject an effective amount of a pharmaceutical composition provided herein (e.g., a liposomal composition), thereby increasing the delivery of oxygen to the tissues and/or organs of the subject. In some embodiments, the subject is elderly (e.g., a human subject that is more than 65, more than 70, more than 75, or more than 80 years of age). In some embodiments, the subject has or is at risk for developing a respiratory condition or disease (e.g., COPD, respiratory distress syndrome or adult respiratory distress syndrome). In some embodiments, the subject has or is at risk for developing a degenerative disorder, such as dementia or Alzheimer's disease. In some embodiments, the method comprises administering the pharmaceutical composition of any one of [46], [47], [100] or [108]) to the subject. Use of a pharmaceutical composition provided herein (e.g., the pharmaceutical composition of any one of [46], [47], [100] or [108]), in the manufacture of a medicament for increasing the delivery of oxygen in an elderly subject is also provided herein. As are, pharmaceutical compositions of any one of [46], [47], [100] or [108] for use in a medical medicament.

In additional embodiments, the disclosure provides a method for increasing the delivery of oxygen in a subject who has or is at risk for developing ischemia/reperfusion injury, that comprises administering to the subject an effective amount of a pharmaceutical composition provided herein, such as liposomal a composition, thereby increasing the delivery of oxygen to the tissues and/or organs in the subject. In some embodiments, the pharmaceutical composition is administered to the subject before, during or following surgery (e.g., transplantation; reattachment of severed extremities, body parts or soft tissues; graft surgery, and vascular surgery). In some embodiments, the ischemia/reperfusion injury is due to a condition selected from infarction, atherosclerosis, thrombosis, thromboembolism, lipid-embolism, bleeding, stent, surgery, angioplasty, end of bypass during surgery, organ transplantation, total ischemia, and combinations thereof. In some embodiments, the ischemia/reperfusion injury is produced in an organ or a tissue selected from the group: heart, liver, kidney, brain, intestine, pancreas, lung, skeletal muscle and combinations thereof. In some embodiments, the ischemia/reperfusion injury is selected from the group: organ dysfunction, infarct, inflammation, oxidative damage, mitochondrial membrane potential damage, apoptosis, reperfusion-related arrhythmia, cardiac stunning, cardiac lipotoxicity, ischemia-derived scar formation, and combinations thereof. In particular embodiments, the ischemia/reperfusion injury is due to myocardial infarction. In some embodiments, the pharmaceutical composition is administered to a subject who has or is at risk for developing peripheral vascular disease, coronary artery disease, stroke, thrombosis, a clot, chronic vascular obstruction or vasculopathy (e.g., secondary to diabetes, hypertension, or peripheral vascular disease), or cerebral ischemia, pulmonary hypertension (adult or neonate); sickle cell disease; neointimal hyperplasia or restenosis (following angioplasty or stenting). In some embodiments, the pharmaceutical composition is administered to a subject who has or is at risk for developing a myopathy, kidney disease; asthma or adult respiratory distress syndrome; Alzheimer's and other dementias secondary to compromised cranial blood flow. In some embodiments, the method comprises administering the pharmaceutical composition of any one of [46], [47], [100] or [108]) to the subject. Use of a pharmaceutical composition provided herein (e.g., the pharmaceutical composition of any one of [46], [47], [100] or [108]), in the manufacture of a medicament for increasing the delivery of oxygen in a subject is also provided herein. As are, pharmaceutical compositions of any one of [46], [47], [100] or [108] for use in a medical medicament.

The pharmaceutical compositions provided herein such as liposomal compositions, have uses that provide advances over prior treatments of diseases and disorders that include without limitation, infection and infectious diseases such as HIV/AIDS: human immunodeficiency virus-1 (HIV-1), tuberculosis, malaria and its complications such as cerebral malaria, severe anemia, acidosis, acute kidney failure and ARDS, sepsis, inflammation (e.g., chronic inflammatory diseases), ischemia, (including an ischemic condition such as ischemic stroke, coronary artery disease, peripheral vascular disease, cerebral vascular disease, ischemia associated renal pathologies, and ischemia associated with wounds); shock (e.g., hemorrhagic shock), stroke, cardiovascular disease, renal pathologies, wound healing, metabolic disease, hyperproliferative diseases such as cancer, and disorders of the immune system, cardiovascular system, digestive, nervous, respiratory, and endocrine system. In some embodiments, the disclosure provides a method for treating or preventing a disease, disorder or condition in a subject needing such treatment or prevention, the method comprising administering an effective amount of a pharmaceutical composition provided herein (e.g., the pharmaceutical composition of any one of [46], [47], [100] or [108]) to the subject. Use of a pharmaceutical composition provided herein (e.g., the pharmaceutical composition of any one of [46], [47], [100] or [108]), in the manufacture of a medicament for the treatment of a disease, disorder or condition in a subject is also provided herein. As are, pharmaceutical compositions of any one of [46], [47], [100] or [108] for use in a medical medicament.

In some embodiments, the disclosure provides a method for treating or preventing a disease, disorder or condition associated with endotoxemia in a subject needing such treatment or prevention, the method comprising administering an effective amount of a pharmaceutical composition provided herein (e.g., the pharmaceutical composition of any one of [46], [47], [100] or [108]) to the subject.

In some embodiments, the disclosure provides a method for treating or preventing a disease, disorder or condition associated with sepsis in a subject needing such treatment or prevention, the method comprising administering an effective amount of a pharmaceutical composition provided herein (e.g., the pharmaceutical composition of any one of [46], [47], [100] or [108]) to the subject. In some embodiments, the subject has a low grade endotoxemic disease.

In some embodiments, the disclosure provides a method for treating or preventing a subject at risk of developing sepsis, the method comprising administering an effective amount of a pharmaceutical composition provided herein (e.g., the pharmaceutical composition of any one of [46], [47], [100] or [108]) to the subject. In some embodiments, the subject is immunocompromised or immunosuppressed. In some embodiments, the subject is critically ill. In some embodiments, the subject elderly or neonatal. In some embodiments, the subject has febrile neutropenia. In some embodiments, the subject has an infection.

In some embodiments, the disclosure provides a method for treating or preventing a disease, disorder or condition associated with burn injury in a subject that is a burn victim, the method comprising administering an effective amount of a pharmaceutical composition provided herein (e.g., the pharmaceutical composition of any one of [46], [47], [100] or [108]) to the subject.

In some embodiments, the disclosure provides a method for treating or preventing a disease, disorder or condition associated with infection in a subject needing such treatment or prevention, the method comprising administering an effective amount of a pharmaceutical composition provided herein (e.g., the pharmaceutical composition of any one of [46], [47], [100] or [108]) to the subject. In some embodiments, the infection is a bacterial infection (e.g., a P. aeruginosa infection, an S. aureus infection (e.g., MRSA), Mycobacterium tuberculosis infection, an enterococcal infection (e.g., VRE), or a condition associated therewith. In some embodiments, the infection is a fungal infection (e.g., a candidiasis infection such as invasive candidiasis) or a condition associated therewith. In some embodiments, the infection is a parasitic infection (e.g., Schistosomiasis, and human African trypanosomiasis), or a condition associated therewith. In some embodiments, the infection is malaria or a condition associated therewith, such as cerebral malaria, severe anemia, acidosis, acute kidney failure and ARDS. In some embodiments, the infection is a viral infection (e.g., Ebola, Dengue and Marburg) or a condition associated therewith, such as influenza, measles, and a viral hemorrhagic fever.

In some embodiments, the disclosure provides a method for treating or preventing a disease, disorder or condition associated with ischemia or hypoxia in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or condition associated with ischemia or hypoxia is associated with surgery or traumatic injury. In some embodiments, the disease or condition is ischemic-reperfusion injury, transient cerebral ischemia, cerebral ischemia-reperfusion, ischemic stroke, hemorrhagic stroke, traumatic brain injury, ischemic heart disease, migraine (e.g., a chronic migraine or severe migraine disorder), gastrointestinal ischemia, kidney disease, pulmonary embolism, acute respiratory failure, neonatal respiratory distress syndrome, obstetric emergencies to reduce perinatal comorbidity (such as, pre/eclampsia and conditions that lead to cerebral palsy), myocardial infarction, acute limb or mesenteric ischemia, cardiac cirrhosis, chronic peripheral vascular disease, congestive heart failure, atherosclerotic stenosis, anemia, thrombosis, embolism, macular degeneration, a neurodegenerative disease (e.g., Alzheimer's disease, Parkinson's disease, and Amyotrophic Lateral Sclerosis (ALS)), sleep apnea, and surgery or traumatic injury. In some embodiments, the disease or condition associated with ischemia or hypoxia is myocardial infarction, or congestive heart failure with or without cardiac cirrhosis. In some embodiments, the disease or condition is pulmonary embolism, acute respiratory failure, chronic peripheral vascular disease, atherosclerotic stenosis, anemia, thrombosis, or embolism. In some embodiments, the disease or condition associated with ischemia or hypoxia is macular degeneration or an oncologic condition associated with hypoxia. In some embodiments, the disease or condition is kidney disease. In some embodiments, the disease or condition is lipopolysaccharide medication or toxin induced acute kidney injury (AKI) or end stage kidney disease.

In some embodiments, the disclosure provides a method for treating or preventing a disease, disorder or condition associated with shock in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or condition is associated with cardiogenic shock. In some embodiments, the disease or condition is associated with, hypovolemic shock. In some embodiments, the disease or condition is associated with septic shock or other forms of distributive shock. In some embodiments, the disease or condition is associated with neurogenic shock. In some embodiments, the disease or condition is associated with anaphylactic shock.

In some embodiments, the disclosure provides a method for treating or preventing a disease, disorder or condition associated with nitric oxide deficiency in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or disorder is sickle cell disease, paroxysmal nocturnal hemoglobinuria (PNH), a hemolytic anemia, a thalassemia, another red blood cell disorder, or a condition associated therewith. In some embodiments, the disease or disorder is a purpura such as thrombotic thrombocytic purpura (TTP), hemolytic uremic syndrome (HUS), idiopathic thrombocytopenia (ITP), or and another platelet disorder, or a condition associated therewith. In some embodiment, the disease or disorder is a coagulation abnormality such as disseminated intravascular coagulopathy (DIC), purpura fulminans, heparin induced thrombocytopenia (HIT), hyperleukocytosis, hyper viscosity syndrome, or a condition associated therewith.

In some embodiments, the disclosure provides a method for treating or preventing a disease, disorder or condition associated with inflammation in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or condition associated with inflammation is low-grade inflammation. In some embodiments, the disease or condition associated with inflammation is systemic inflammation. In some embodiments, the disease or condition associated with inflammation is acute inflammation or a chronic inflammatory disease.

In some embodiments, the disclosure provides a method for treating or preventing a disease, disorder or condition associated with a cardiovascular disease or condition in a subject needing such treatment or prevention, the method comprising administering an effective amount of a pharmaceutical composition provided herein (e.g., the pharmaceutical composition of any one of [46], [47], [100] or [108]) to the subject. In some embodiments, cardiovascular disease or condition is coronary artery disease. In some embodiments the cardiovascular disease or condition is myocardial infarction, sudden cardiac death, cardiorespiratory arrest, hypertension, pulmonary arterial hypertension, atherosclerosis, occlusive arterial disease, Raynaud's disease, peripheral vascular disease, other vasculopathies such as Buerger's disease, Takayasu's arthritis, and post-cardiac arrest syndrome (PCAS), chronic venous insufficiency, heart disease, congestive heart failure, or a chronic skin ulcer. Methods and biomarkers for evaluating cardiovascular health (e.g., levels of conventional troponins (cTnI and cTnT), Ischemia-Modified Albumin (IMA), B-type Natriuretic Peptide and N-terminal proBNP, whole blood choline, and unesterified free fatty acid (FFAu)) and cardiovascular injury and disease, and the efficacy of treatment regimens are known in the art

In some embodiments, the disclosure provides a method for treating or preventing a disease, disorder or condition associated with a liver disease, injury or condition in a subject needing such treatment or prevention, the method comprising administering an effective amount of a pharmaceutical composition provided herein (e.g., the pharmaceutical composition of any one of [46], [47], [100] or [108]) to the subject. In some embodiments, the liver disease or condition is hepatic ischemia/reperfusion injury. In some embodiments, the liver disease or condition is a hepatic resection or liver transplantation. In some embodiments, the liver disease or condition is cirrhosis. In some embodiments, the liver disease or condition is nonalcoholic fatty liver disease (NAFLD), non-alcoholic steatohepatitis (NASH). In some embodiments, the liver disease or condition is alcoholic liver disease. In some embodiments, the liver disease or condition is acute liver injury. Methods and biomarkers for evaluating liver health (e.g., levels of liver enzymes ALT, AST, ALP, and LDH), as well as liver injury and disease and the efficacy of treatment regimens are known in the art.

In some embodiments, the disclosure provides a method for treating or preventing a disease, disorder or condition associated with a lung disease or condition in a subject needing such treatment or prevention, the method comprising administering an effective amount of a pharmaceutical composition provided herein (e.g., the pharmaceutical composition of any one of [46], [47], [100] or [108]) to the subject. In some embodiments, the lung disease or condition is acute respiratory distress syndrome (ARDS). In some embodiments, the lung disease or condition is chronic obstructive pulmonary disease. In some embodiments, the lung disease or condition is pulmonary fibrosis. In some embodiments, the lung disease or condition is pulmonary hemorrhage. In some embodiments, the lung disease or condition is asthma. In some embodiments, the lung disease or condition is lung injury. In some embodiments, the lung disease or condition is lung cancer. In some embodiments, the condition is cystic fibrosis.

In some embodiments, an effective amount of a pharmaceutical composition provided herein (e.g., the pharmaceutical composition of any one of [46], [47], [100] or [108])) is administered to a subject (e.g., a human) experiencing acute lung distress (e.g., presenting symptoms such as having difficulty breathing, tachypnea, mental confusion due to low oxygen levels) and/or having a PaO2/FiO2 ratio of less than 300 mm Hg. In additional embodiments, the pharmaceutical composition is administered to a subject having a PaO2/FiO2 ratio of <300 mm Hg to ≥200 mm Hg. In further embodiments the pharmaceutical composition comprises trans-crocetin.

In some embodiments, an effective amount of a pharmaceutical composition provided herein (e.g., the pharmaceutical composition of any of [1]-[79]) is administered to a subject (e.g., a human) experiencing Acute Respiratory ARDS and/or having a PaO2/FiO2 ratio of less than 200 mm Hg. In further embodiments the pharmaceutical composition comprises liposomal trans-crocetin.

In some embodiments, an effective amount of a pharmaceutical composition provided herein (e.g., the pharmaceutical composition of any one of [46], [47], [100] or [108])]) is administered to a subject (e.g., a human) in order to increase the patient's PaO2/FiO2 ratio. In some embodiments, administration of the pharmaceutical composition increases the patient's PaO2/FiO2 ratio by at 5%-75%, or any range therein between. In further embodiments, the administration of the pharmaceutical composition increases the patient's PaO2/FiO2 ratio by at least 5%, 10%, 15%, 20%, 25%, 30% 40% or 50%. In further embodiments the pharmaceutical composition comprises trans-crocetin. In further embodiments, the administration of the trans-crocetin increases the patient's PaO2/FiO2 ratio by at 5%-75%, or any range therein between. In further embodiments, the administration of the trans-crocetin increases the patient's PaO2/FiO2 ratio by at least 5%, 10%, 15%, 20%, 25%, 30% 40% or 50%.

In some embodiments, the disclosure provides a method for treating or preventing a disease, disorder or condition associated with a kidney disease or condition in a subject needing such treatment or prevention, the method comprising administering an effective amount of a pharmaceutical composition provided herein (e.g., the pharmaceutical composition of any one of [46], [47], [100] or [108]) to the subject. In some embodiments, the kidney disease or condition is lipopolysaccharide-induced acute kidney injury (AKI). In some embodiments, the kidney disease or condition is chronic renal failure with or without end stage kidney disease. Methods and biomarkers for evaluating renal health (e.g., levels of N-acetyl-β-glucosaminidase (NAG), α₁-microglobulin (α₁M), Cystatin-C(Cys-C), Retinol binding protein (RBP), microalbumin, Kidney injury molecule-1 (KIM-1), Clusterin, Interleukin-18 (IL-18), Cysteine-rich protein (Cyr61), osteopontin (OPN), Fatty acid-binding protein (FABP), Fetuin-A, and neutrophil gelatinase-associated lipocalin (NGAL)), as well as renal injury and disease and the efficacy of treatment regimens are known in the art.

In some embodiments, the disclosure provides a method for treating or preventing a disease, disorder or condition associated with a vascular disease in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or condition is coronary artery disease. In some embodiments, the disease or condition is hypertension. In some embodiments, the disease or condition is atherosclerosis. In some embodiments, the disease or condition is post-cardiac arrest syndrome (PCAS). In some embodiments, the disease or condition is occlusive arterial disease, peripheral vascular disease, chronic venous insufficiency, chronic skin ulcers, or Raynaud's disease. In some embodiments, the disease, disorder or condition associated with a vascular disease is heart disease. In further embodiments, the disease, disorder or condition is congestive heart failure. In some embodiments, the disease, disorder or condition associated with vascular disease is ischemic bowel disease.

In some embodiments, the disclosure provides a method for treating or preventing a disease, disorder or condition associated with a heart attack or stroke in a subject needing such treatment or prevention and/or at risk of having a heart attack or stroke, the method comprising administering an effective amount of a pharmaceutical composition provided herein (e.g., the pharmaceutical composition of any one of [46], [47], [100] or [108]) to the subject. In some embodiments, the disease, disorder or condition is ischemic stroke. In some embodiments, the disease, disorder or condition is hemorrhagic stroke. Methods and biomarkers for evaluating heart attack and stroke (e.g., levels of blood B-type natriuretic peptide (BNP), C-reactive protein (CRP), GlycA, CK-MB, Cardiac troponin, myoglobin, low-density lipoprotein-cholesterol and hemoglobin A1c (HgA1c), lipoprotein-associated phospholipase A2, glial fibrillary acidic protein, S100b, neuron-specific enolase, myelin basic protein, interleukin-6, matrix metalloproteinase (MMP)-9, D-dimer, and fibrinogen)), and the efficacy of treatment regimens are known in the art.

In some embodiments, the disclosure provides a method for treating or preventing a disease, disorder or condition associated with nervous system in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease or condition is pain (e.g., chronic pain). In some embodiments, the disease or condition is a neurodegenerative disease (e.g., Alzheimer's disease or Parkinson's disease).

In some embodiments, the disclosure provides a method for treating or preventing a disease, disorder or condition associated with inflammatory bowel disease in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease, disorder or condition is Crohn's disease. In some embodiments, the disease, disorder or condition is ulcerative colitis.

In some embodiments, the disclosure provides a method for treating or preventing a disease, disorder or condition associated with type 2 diabetes or predisposition for diabetes in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease, disorder or condition is metabolic disease. In some embodiments, the disease, disorder or condition is insulin resistance. In some embodiments, the disease, disorder or condition is a diabetic vascular disease (e.g., a microvascular disease such as retinopathy and nephropathy). In some embodiments, the disease, disorder or condition is diabetic neuropathy. In some embodiments, the disease, disorder or condition is ulcers, diabetic necrosis, or gangrene.

In some embodiments, the disclosure provides a method for treating or preventing a myopathy, chronic microvascular disease, or microangiopathy, or a disorder associated with microvascular dysfunction such as age-related macular degeneration (AMD) in a subject needing such treatment or prevention, the method comprising administering an effective amount of a pharmaceutical composition provided herein (e.g., the pharmaceutical composition of any one of [46], [47], [100] or [108]) to the subject.

In some embodiments, the disclosure provides a method for treating or preventing a disease, disorder or condition associated with sclerosis in a subject needing such treatment or prevention, the method comprising administering an effective amount of a pharmaceutical composition provided herein (e.g., the pharmaceutical composition of any one of [46], [47], [100] or [108]) to the subject. In some embodiments, the disease, disorder or condition associated with sclerosis is systemic sclerosis.

In some embodiments, the disclosure provides a method for treating endotoxemia in a subject needing such treatment, the method comprising administering an effective amount of a pharmaceutical composition provided herein (e.g., the pharmaceutical composition of any one of [46], [47], [100] or [108]) to the subject. In some embodiments, the endotoxemia is associated with a condition such as periodontal disease (e.g., periodontitis or inflammation of the gums), chronic alcoholism, chronic smoking, transplantation, or neonatal necrotizing enterocolitis, or neonatal ear infection.

In some embodiments, the disclosure provides a method of reducing systemic levels of LPS, endotoxin and/or another trigger of systemic inflammation in a subject in need thereof, the method comprising administering an effective amount of a pharmaceutical composition provided herein (e.g., the pharmaceutical composition of any one of [46], [47], [100] or [108]) to the subject.

### Combination Therapy

The compositions provided herein can be administered alone or in combination therapy with one or more additional therapeutic agents. In some embodiments, the composition is administered in combination therapy with another therapeutic agent. Combinations may be administered either concomitantly, e.g., combined in the same liposomal composition, delivery vehicle (e.g., liposome), as an admixture, separately but simultaneously or concurrently; or sequentially. This includes presentations in which the combined therapeutic agents are administered together as a therapeutic mixture, and also procedures in which the combined agents are administered separately but simultaneously, e.g., as through separate intravenous lines into the same individual. Administration “in combination” further includes the separate administration of one of the therapeutic agents given first, followed by the second. Methods of treatment using the combination therapy are also provided.

In additional embodiments, a composition provided herein is administered in combination with another therapeutic agent. In some embodiments, a pharmaceutical composition comprising an aqueous solution of any one of [1]-[45], [98], [99], [106], or [107] is administered in combination with another therapeutic agent. In some embodiments, an aqueous solution of any one of [1]-[45], [98], [99], [106], or [107] comprising a salt of a carotenoid provided in any of FIG. 1A-FIG. 1D is administered in combination therapy with another therapeutic agent. In some embodiments, a composition comprising a multivalent salt (e.g., a monovalent salt or a trivalent salt) of a carotenoid provided in any of FIG. 1A-FIG. 1D herein, is administered in combination therapy with another therapeutic agent. In particular embodiments, a composition comprising a multivalent salt of trans-crocetin (e.g., CTC or MTC) is administered in combination therapy with another therapeutic agent. In other particular embodiments, a composition comprising a multivalent salt of trans-norbixin (e.g., CTN or MTN) is administered in combination therapy with another therapeutic agent.

In some embodiments, a pharmaceutical composition comprising a salt of one or more ionizable carotenoids is administered in combination therapy with a carotenoid comprising at least one polar group or monocyclic group. In some embodiments, the salt of the ionizable carotenoid is a multivalent salt (e.g., salt containing monovalent, trivalent or tetravalent counterion). In some embodiments the ionizable carotenoid is a carotenoid contained in the aqueous solution of any one of [1]-[45], [98], [99], [106], or [107] and/or FIG. 1A-FIG. 1D. In one embodiment, the carotenoid comprising at least one polar group or monocyclic group polar group is symmetric. In another embodiment, a monovalent ionizable carotenoid salt composition is administered in combination therapy with at least one carotenoid selected from: zeanthin, astaxanthin, lutein, and xanthophyll. In another embodiment, the monovalent ionizable carotenoid salt composition is administered in combination therapy with astaxanthin. In another embodiment, the carotenoid comprising at least one polar group or monocyclic group polar group is asymmetric. In another embodiment, a monovalent ionizable carotenoid salt composition disclosed herein is administered in combination abscisic acid (ABA).

In some embodiments, a pharmaceutical composition comprising an ionizable carotenoid salt provided herein is administered in combination therapy with a standard of care treatment for the disease, disorder, or condition to be treated. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an antimicrobial agent. In some embodiments, the antimicrobial agent is an anti-bacterial agent. In some embodiments, the antibacterial agent is selected from, but not limited to, ertapenem, piperacillin-tazobactam, cefepime, aztreonam, metronidazole, meropenem, ceftriaxone, ciprofloxacin, vancomycin, linezolid, tobramycin, levofloxacin, azithromycin, cefazolin, and ampicillin. In some embodiments, the antibacterial agent is selected from, but not limited to, ceftriaxone, levofloxacin, ciprofloxacin, cefazolin, piperacillin-tazobactam, meropenem, metronidazole, vancomycin, and ampicillin. In other embodiments, the antimicrobial agent is an anti-fungal agent. In further embodiments, the anti-fungal agent is caspofungin or another antifungal drug. In other embodiments, the antimicrobial agent is an anti-malarial agent. In further embodiments, the anti-malarial agent is selected from, but not limited to, artemisinin and its analogs, chloroquin and its analogs, atovaquone, a quinine derivative, proguanil or another anti-malarial drug. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with activated protein C (e.g., rhAPC), or drotrecogin alfa (activated) (DAA). In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a corticosteroid (e.g., a glucocorticoid or mineralocorticoid such as fludrocortisonel). In some embodiments, the corticosteroid is a glucocorticoid. In further embodiments, the glucocorticoid is selected from cortisone, ethamethasoneb, prednisone, prednisolone, triamcinolone, dexamethasone and methylprednisolone. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with intravenous administration of a vitamin. In some embodiments, the vitamin is vitamin C (ascorbic acid). In some embodiments, the vitamin is vitamin A. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a glucocorticoid and vitamin C (e.g., intravenous vitamin C administration). In some embodiments, the glucocorticoid is selected from cortisone, ethamethasoneb, prednisone, prednisolone, triamcinolone, dexamethasone and methylprednisolone. In further embodiments, the glucocorticoid is hydrocortisone. In additional embodiments, at least one ionizable carotenoid composition provided herein (e.g., a monovalent or bivalent salt composition comprising an ionizable carotenoid disclosed in FIG. 1A, FIG. 1B, FIG. 1C, and/or FIG. 1D) is administered in combination therapy with a glucocorticoid, vitamin C, and thiamine. In some embodiments, the salt of the ionizable carotenoid is a monovalent salt. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], or [99], and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a vasopressor agent. In some embodiments, the vasopressor therapeutic agent is norepinephrine or similar drugs, or angiotensin II (e.g., GIAPREZA™). In some embodiments, the vasopressor therapeutic agent is epinephrine, phenylnephrine, dopamine, or vasopressin. In some embodiments, the vasopressor therapeutic agent is ephedrine, milrinone, isoproterenol, dobutamine, isoproterenol, or dopamine.

In some embodiments, a pharmaceutical composition comprising an ionizable carotenoid salt provided herein is administered in combination therapy with a thrombolytic therapeutic agent. In some embodiments, the thrombolytic therapeutic agent tissue plasminogen activator (tPA). In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In additional embodiments, a pharmaceutical composition comprising an ionizable carotenoid salt provided herein is administered in combination therapy with an anesthetic agent. In some embodiments, the anesthetic agent is administered before the pharmaceutical composition (e.g., as an anesthetic preconditioning (APC) regimen, prior to surgery). In some embodiments, the anesthetic agent is administered after the pharmaceutical composition (e.g., post-surgery). In some embodiments, anesthetic agent is isoflurane, sevoflurane, or propofol. In some embodiments, anesthetic agent is cystathionine-β-synthase (CBS), cystathionine-γ-lyase (CSE), or 3-mercapto-pyruvate-sulfur-transferase (MST). In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a therapeutic agent. In some embodiments, a pharmaceutical composition of any of [1]-[28] is administered in combination with a therapeutic agent. In some embodiments, a pharmaceutical composition comprising a multivalent salt of a carotenoid provided in any of FIG. 1A-FIG. 1D herein, is administered in combination therapy with a therapeutic agent. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with a therapeutic agent selected from: heparin, vasopressin, antidiuretic hormone (ADH), and a 3-Hydroxy-3-methylglutaryl coenzyme A reductase inhibitor (statin). In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an anti-inflammatory therapeutic agent. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with oxygen and/or intravenous fluids to maintain/increase blood oxygen levels and/or blood pressure or hyperbaric therapy. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with an antioxidant. In some embodiments, the ionizable carotenoid salt provided herein is administered in combination therapy with at least one of alpha-tocopherol, melatonin, ascorbic acid (AA), alpha lipoic acid, desferoxamine, and trimetazidine (TMZ). In some embodiments, the ionizable carotenoid salt provided herein is administered in combination therapy with at least one of glutatione, N-Acetylcysteine (NAC), Bucillamine (N-(2-mercapto-2-methylpropionyl)-1-cysteine), a superoxide dismutase (SOD) or derivative thereof, catalase (CAT), and allopurinol, idebenone. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the administered ionizable carotenoid is trans-crocetin (e.g., CTC or STC). In other particular embodiments, the administered ionizable carotenoid is trans-norbixin (e.g., CTN and STN).

In some embodiments, a pharmaceutical composition comprising an ionizable carotenoid salt provided herein is administered in combination therapy with a chemotherapeutic agent (e.g., to enhance the effect of chemotherapy on cancer cells and mitigate the effects of chemotherapy-induced myelosuppression and anemia). In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with immunotherapy. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

In some embodiments, a pharmaceutical composition comprising an ionizable carotenoid salt provided herein is administered in combination therapy with radiotherapy. In some embodiments, the salt of the ionizable carotenoid is a multivalent salt (e.g., bivalent, trivalent or tetravalent). In some embodiments the ionizable carotenoid is a carotenoid contained in the aqueous solution of any one of [1]-[45], [98], [99], [106], or [107] and/or FIG. 1A-FIG. 1D. In particular embodiments, the ionizable carotenoid is trans-crocetin (e.g., CTC or STC). In other particular embodiments, the ionizable carotenoid is trans-norbixin (e.g., CTN and STN).

### Kits for Administration of Active Agents

In another embodiments, the disclosure provides a kit for administering a pharmaceutical composition provided herein (e.g., the pharmaceutical compositions according to any one of [46], [47], [100], [108]) to a subject for treating a disease, disorder, or condition. In some embodiments, the disclosure provides a kit for delivering a therapeutic agent to a subject, the kit comprising: (a) a first composition comprising a disclosed aqueous solution (e.g., the aqueous solution according to any one of [1]-[45], [98], [99], [106], or [107]) and (b) a second composition containing for example, reagents, buffers, excipients, or another therapeutic agent that is stored separately prior to administration to the subject. Such kits typically include two or more components necessary for treating a disease state, such as hypoxia or inflammation related condition. In some embodiments, the kits include for example, a provided aqueous solution, compositions, reagents, buffers, containers and/or equipment. In some embodiments, the kits include a packaging assembly that include one or more components used for treating the disease state of a patient. For example, a packaging assembly may include separate containers that house the reagents necessary to formulate the aqueous solutions and other excipients or therapeutic agents that can be mixed with the compositions prior to administration to a patient. In some embodiments, a physician may select and match certain components and/or packaging assemblies depending on the treatment or diagnosis needed for a particular patient.

### EXAMPLES

### Example 1—Aqueous Formulation of Trans Sodium Crocetinate (TSC)

The following presents exemplary aqueous solutions provided herein and a process for preparing a TSC pharmaceutical composition

A TSC pharmaceutical composition containing 2 mgml TSC, 45 mg/ml PEG400 and 30 mg/ml sucrose was prepared according to the following protocol. The prepreared pharmaceutic composition was a sterile, clear, red solution in 10 mL amber glass, single-use vials. Each vial contains 20 mg of TSC, 450 mg PEG400 and 300 mg of sucrose.

### Formulation Process:

TABLE 1
Formulation of 55 L of L4L-027

| | | |
|---|---|---|
| Scale | 55 | L |
| Density | 1.015 | Kg/L |
| Total weight | 55.825 | Kg |
| Sucrose | 1650 | gram |
| PEG400 | 2475 | gram |
| TSC | 110 | gram |
| Water | 51.59 | Kg |

1650 grams of sucrose was added to 51.59 Kg of WFI with mixing in a Nalgene tank containing a tank liner. 2475 grams of PEG400 was then added to the tank with mixing. After the sucrose and PEG400 were completely dissolved, 110 grams of TSC was added and the solution was mixed at room temperature for a minimum of 30 minutes or until the solution was clear. The solution was then filtered through a 0.45 μm glass fiber prefilter+0.2 μm sterile filter at a pump speed of 700 mL/min into a 100 L Biobag. A sample of the filtrate was pulled from the Biobag solution for a bioburden test. The Biobag was stored at 2-8° C. for at least 12 hours and then brought to room temperature and sterile filtered through 0.45 μm glass fiber prefilter+0.2 μm sterile filter at a pump speed of 700 mL/min to a 100 L Biobag. The sterile filtrate was then filled into amber 10R vials with a target 10.5 ml fill volume.

Other exemplary TSC aqueous solutions were formulated with varying concentrations of PEG400, NaCl, sucrose and TSC concentrations. The results are summarized in Table 2.

### Observations:

All formulations could be carried out at room temperature with mixing. The dissolution of TSC was generally completed within 10 minutes. No TSC was observed to crash out of the solution upon ten times dilution in 0.9% saline for 2 mg/mL formulation. No TSC was observed to crash out of solution after storage at 2-8° C. The solution pH is around 8.7 and no further pH adjustment was needed. The final formulation has an osmolality around 270 mOsm.

TABLE 2
Additional Exemplary Prepared Formulations

| PEG400 | NaCl | Sucrose | TSC Concentration | |
|---|---|---|---|---|
| (v/v) | (w/w) | (w/v) | (mg/mL) | Observation |
| 80% | 0% | 0% | 20 | soluble |
| 8% | 0% | 0% | 8 | saturated with some |
| | | | | insoluble TSC |
| 6% | 0% | 0% | 4 | soluble |
| 4% | 0% | 0% | 4 | soluble |
| 2% | 0% | 0% | 2 | soluble |
| 4% | 0.9% | 0% | 4 | partially soluble |
| 4% | 0.9% | 0% | 2 | partially soluble |
| 4% | 0.9% | 0% | 2 | soluble |
| 4% | 0% | 3% | 2 | soluble |

### Example 2—Preparation of Calcium Acetate Liposomes with Nanoassemblr®

Calcium acetate loaded liposomes were prepared by the following procedure. First, the lipid components of the liposome lipid membrane were weighed out and combined as a concentrated solution in ethanol at a temperature of around 65° C. In one example, the lipids used were hydrogenated soy phosphatidylcholine, cholesterol, and DSPE-PEG-2000 (1,2-distearoyl-sn-glycero-3-phosphoethanolamine-N-[methoxy-(polyethylene glycol)-2000]).

The molar ratio of HSPC:cholesterol:PEG-DSPE was approximately 3:2:0.15. In another example, the lipids used were HSPC, cholesterol, PEG-DSPE-2000, and 1-palmitoyl-2-glutaryl-sn-glycero-3-phosphocholine (PGPC). The molar ratio of HSPC:cholesterol:PEG-DSPE:PGPC was approximately 2.7:2:0.15:0.3.

Next, calcium acetate was dissolved in an aqueous buffer at a concentration of 125 or 250 mM, with a pH of 7.0. The calcium acetate solution was heated to 65° C. The ethanolic lipid solution and the calcium acetate solution were separately transferred to syringes. Two solutions were injected into microfluidic channel and mixed while flowing through it with Precision NanoSystems' NanoAssemblr® device. The mixing was performed at an elevated temperature (63° C.-72° C.) to ensure that the lipids were in the liquid crystalline state (as opposed to the gel state that they would attain at temperatures below the lipid transition temperature (Tm=51° C.-54° C.)). The size of liposome can be controlled by ratio between lipid solution and aqueous solution, as well as the mixing flow rate.

### Example 3—Efficient Method of Loading Ionizable Carotenoids into Liposome

The following approach provides a highly efficient method for loading ionizable carotenoids and other low solubility compounds into liposome using short chain polyethylene glycol as the helper solvent.

### Process Description

A solution of 4 mg/mL of TSC in 6% PEG400 was mixed with (diafiltrated liposome with calcium acetate gradient) at room temperature. The mixture was then quickly heated using a heat exchanger to 68±5° C. and incubated at 68±5° C. for 10 minutes, followed by quick cool down to no more than 30° C. using an ice water bath. The incubation step can be skipped if the cooling step is carried out immediately in a continuous process.

### Process Description

The advantages of using a short chain polyethylene glycol as a helper solvent in this loading protocol include, inter alia, the ability to rapidly load high concentrations of TSC in liposomes with almost quantitative loading efficiency. Short chain polyethylene glycols having an average molecular weight of 200-600 Da (e.g., PEG-300 and PEF-500) may work as well as PEG400 as a helper solvent in this loading protocol. Short chain PEG concentration useful according to this process include ranges of 0.1% to 20% (w/w), 0.5% to 15% (w/w), or 1% to 10% (w/w). In particular embodiments, the short PEG concentration is 2-7% (w/w). Other ionizable carotenoids such as trans-norbixin and other hydrophobic caroboxylic acids can also be loaded into liposomes using the same strategy or an iteration thereof. For example, the methods can be applied using other monovalent counterions such as NH₄⁺, Li⁺, K⁺, or a monovalent organic cation such as protonated amine. Moreover, the intraliposomal gradient can contain a different weak acid (e.g., an organic acid selected from acetic acid, gluconic acid, tartaric acid, glutamic acid, citric acid, formic acid, and glycinic acid) salt of a multivalent metal (e.g., a bivalent metal selected from a bivalent metal selected from Ca²⁺, Mg²⁺, Zn²⁺, Cu²⁺, Co²⁺, and Fe²⁺), such as magnesium acetate, zinc acetate or other soluble metal ion salts.

### Example 4—Liposomal CTC Efficacy Studies in Mice (Study 1)

The objective of this study was to evaluate the efficacy of liposome encapsulated calcium trans-crocetinate (L4L-121) plus standard of care when compared to saline plus standard of care, in a CLP model used to induce severe grade sepsis. For this study, ten male mice per group with ages ranging from 8-10 weeks were treated for 5 days. The mice in the control group received a daily administration of 0.9% saline via IP injection at a volume of 0.25 mL for 5 days in conjunction with 12.5 mg/kg of imipenem twice daily. The other mice were treated with 50 mg/kg of L4L-121 once daily in conjunction with 12.5 mg/kg of imipenem twice daily.

The study duration was 6 days. The following efficacy parameters were evaluated for all groups:
Blood was collected on all animals following euthanasia
Following blood collection, heart, spleen, liver, lungs, kidneys and cecum were collected.
Survival was assessed by enumeration of mortality over 5 days following CLP surgery.

### Results

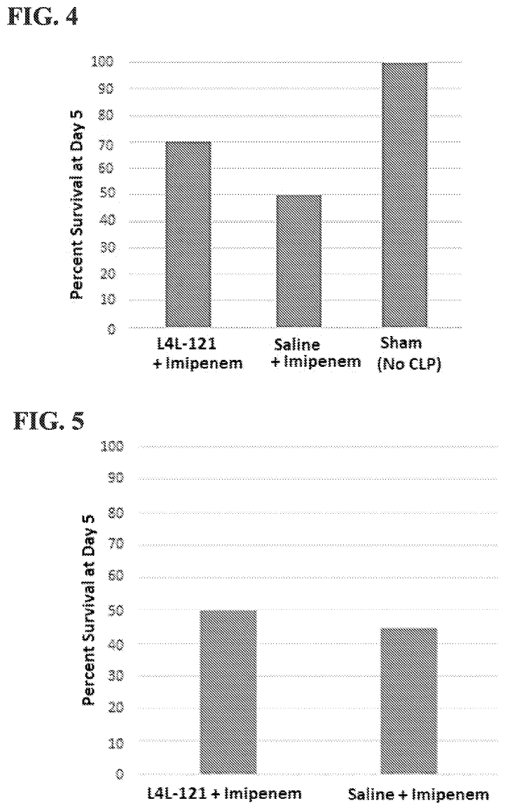

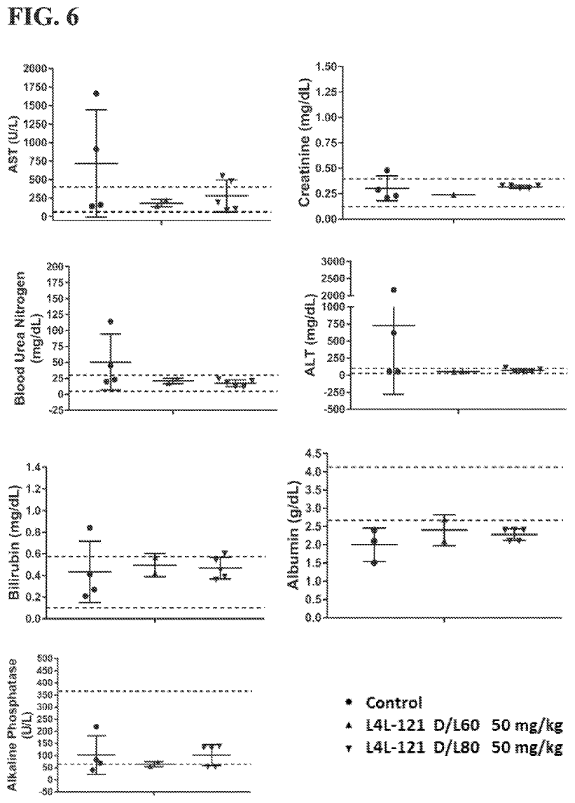

As shown in FIG. 4, although L4L-121 in combination with imipenem demonstrated a reduction in mortality when compared to the saline plus imipenem control group. Thirty percent mortality was observed in the L4L-121 with imipenem group, compared to 50% mortality in the saline with imipenem group.

### L4L-121 Efficacy Study 2: Study Design and Results

### Study Design

This study was performed to evaluate the efficacy of L4L-121 plus standard of care when compared to saline plus standard of care, in a CLP model used to induce severe grade sepsis. For this study, ten male mice in each group with ages ranging from 8-10 weeks were treated for 5 days. The mice in the control group received a daily administration of 0.9% saline via IP injection at a volume of 0.3 mL for 5 days in conjunction with 12.5 mg/kg of imipenem twice daily. The other mice were treated with 50 mg/kg of L4L-121 with a drug to lipid ratio of 60 gm/mol of lipid (D/L 60) once daily in conjunction with 12.5 mg/kg of imipenem twice daily, or 50 mg/kg of L4L-121 with a drug to lipid ratio of 80 gm/mol of lipid (D/L 80) once daily in conjunction with 12.5 mg/kg of imipenem twice daily.

The study duration was 6 days. The following efficacy parameters were evaluated for all groups:
Survival was assessed by enumeration of mortality over 5 days following CLP surgery.
Animals which were found dead had half of each tissue collected (lungs, kidneys, heart, spleen and liver) collected.
Animals that were euthanized prior to study end had blood and tissue collected.
Blood was collected on all animals following euthanasia for both moribund mice and those remaining at Day 5 and a full chemistry panel was performed which included assessment of albumin, alkaline phosphatase (ALP), alanine aminotransferase (ALT), aspartate aminotransferase (AST), blood urea nitrogen (BUN), calcium, cholesterol, creatine kinase, creatinine, glucose, total bilirubin, total protein, triglycerides).

### Results

As shown in FIG. 5, L4L-121 D/L 80 in combination with imipenem demonstrated a reduction in mortality when compared to the imipenem-treated control group. Fifty percent mortality was observed in the L4L-121 D/L 80 group, compared to 60% in the D/L 60 and the saline plus imipenem groups. None of the deaths recorded in the L4L-121 D/L 80 group were attributed to euthanasia while two of the six deaths recorded in the saline group were a result of euthanasia.

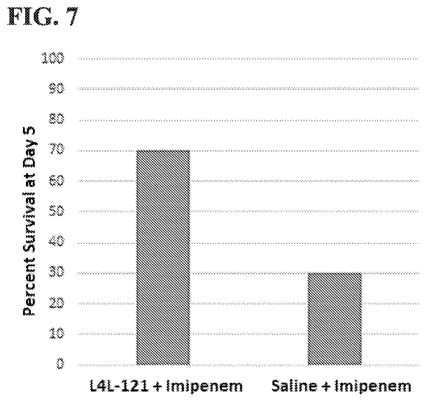

At the time of euthanasia, blood samples, lungs, kidneys, heart, spleen and liver were harvested from the mice. In order to evaluate if any toxicity was present following treatment with L4L-121, a histopathological review of the tissues was performed, and a complete serum chemistry was analyzed. As indicated in FIG. 7, when compared to the saline control group, there were no clinically meaningful changes in creatinine, blood urea nitrogen, alanine aminotransferase, total bilirubin and alkaline phosphatase levels following a daily treatment with L4L-121 at the dose level of 50 mg/kg. There was a slight decrease in albumin levels following treatment with L4L-121 and a numerically greater decrease in the saline group.

### L4L-121 Efficacy Study 3: Study Design and Results

### Study Design

The objective of this study was to evaluate the efficacy of L4L-121 plus standard of care when compared to saline plus standard of care, in a CLP model used to induce severe grade sepsis. For this study, ten female mice per group with ages ranging from 11-12 weeks were treated for 5 days. The mice in the control group received a daily administration of 0.9% saline via IP injection at a volume of 0.3 mL for 5 days in conjunction with 12.5 mg/kg of imipenem twice daily. The other mice were treated with 50 mg/kg of L4L-121 once daily in conjunction with 12.5 mg/kg of imipenem twice daily.

The study duration was 6 days. The efficacy parameters were evaluated for all groups as follows:
Survival was assessed by enumeration of mortality over 5 days following CLP surgery.
Animals which were found dead had half of each tissue collected (lungs, kidneys, heart, spleen and liver) collected.
Animals that were euthanized prior to study end had blood and tissue collected.
Blood was collected on all animals following euthanasia for both moribund mice and those remaining at Day 5 and a full chemistry panel was performed (albumin, ALP, ALT, AST, BUN, calcium, cholesterol, creatine kinase, creatinine, glucose, total bilirubin, total Protein and triglycerides).

### Results

All mice in all treatment groups received all five doses of daily treatment. As seen in FIG. 7, mice treated with L4L-121 and imipenem showed 30% mortality, with two of the three deaths attributed to euthanasia. By contrast, animals treated with saline vehicle and imipenem demonstrated 70% death and one of the seven deaths was a result of euthanasia.

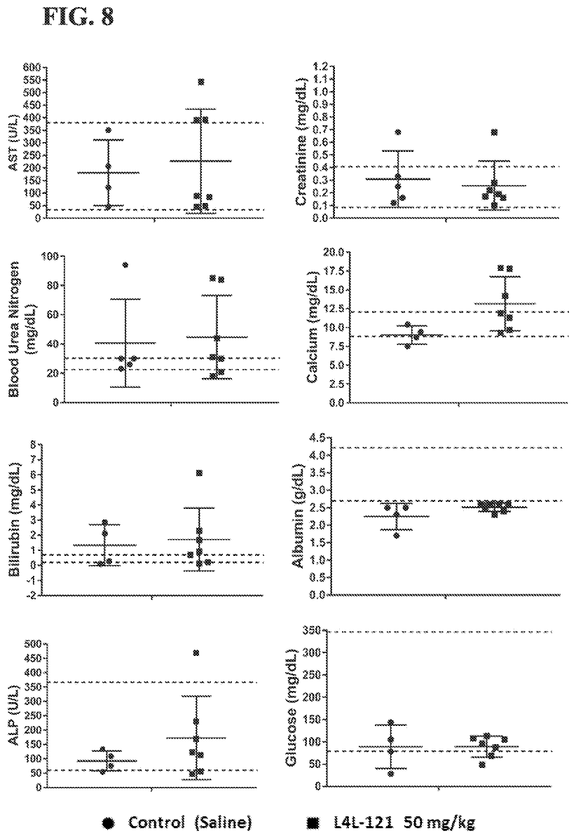

Liver indices were obtained from mice treated with L4L-121 plus imipenem as well as mice treated with saline plus imipenem. As can be seen in FIG. 8, there were no appreciable differences in creatinine, glucose and ALP levels between both groups. Albumin levels were lower than the normal range, both in mice treated with L4L-121 plus imipenem and those treated with saline plus imipenem. There was an increase in blood urea nitrogen and total bilirubin in both groups of mice, and calcium was higher in mice treated with L4L-121 plus imipenem.

### L4L-121 Efficacy Study 4: Study Design and Results

### Study Design

Based on observations from the prior 3 studies, the Sponsor decided to perform a dose ranging study to optimize the L4L-121 dose. The objective of this study was to evaluate the efficacy of varying doses of L4L-121 plus standard of care when compared to saline and standard of care, in a CLP model used to induce severe grade sepsis. For this study, ten female mice per arm, with ages ranging from 9-10 weeks, were treated for 5 days. The mice in the control group received a daily administration of 0.9% saline via IP injection at a volume of 0.3 mL for 5 days in conjunction with 12.5 mg/kg of imipenem twice daily. The other mice were treated with four different doses of LAL-121 once daily (1 mg/kg, 5 mg/kg, 25 mg/kg and 50 mg/kg) in conjunction with 12.5 mg/kg of imipenem twice daily.

The study duration was 6 days. The efficacy parameters were evaluated for all groups as follows:
Survival was assessed by enumeration of mortality over 5 days following CLP surgery.
Animals which were found dead had half of each tissue collected (lungs, kidneys, heart, spleen and liver) collected.
Animals that were euthanized prior to study end had blood and tissue collected.
Blood was collected on all animals following euthanasia for both moribund mice and those remaining at Day 5 and a full chemistry panel was performed (albumin, ALP, ALT, AST, BUN, calcium, cholesterol, creatine kinase, creatinine, glucose, total bilirubin, total protein, triglycerides).

### Results

Data from this study showed that there was a significant improvement in survival following treatment with a 5 mg/kg dose of L4L-121 plus imipenem compared to saline plus imipenem treated animals.

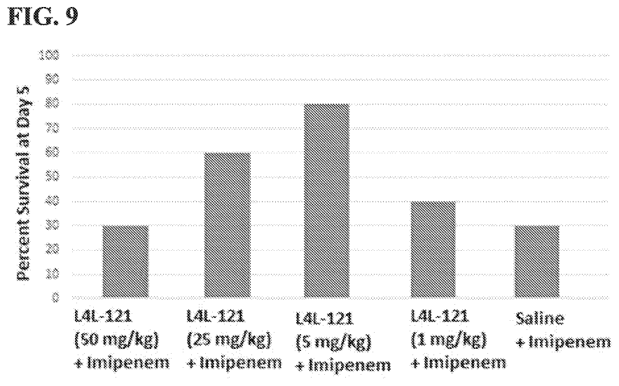

As seen in FIG. 9, mice treated with a 50 mg/kg dose of L4L-121 and imipenem demonstrated 70% mortality. Three of the seven deaths were a result of euthanasia. Mice which received a 25 mg/kg dose of L4L-121 and imipenem had 40% mortality. Two of the four deaths were due to euthanasia. Mice treated with a 5 mg/kg dose of L4L-121 and imipenem had 20% death. None of the deaths were due to euthanasia. Animals treated with saline vehicle and imipenem demonstrated 70% death. Treatment with 5 mg/kg dose of L4L-121 demonstrated a statistically significant decrease in mortality when compared to the vehicle control group (P=0.0321). Mice which received a 1 mg/kg dose of L4L-121 and imipenem had 60% mortality. None of the deaths were attributed to euthanasia.

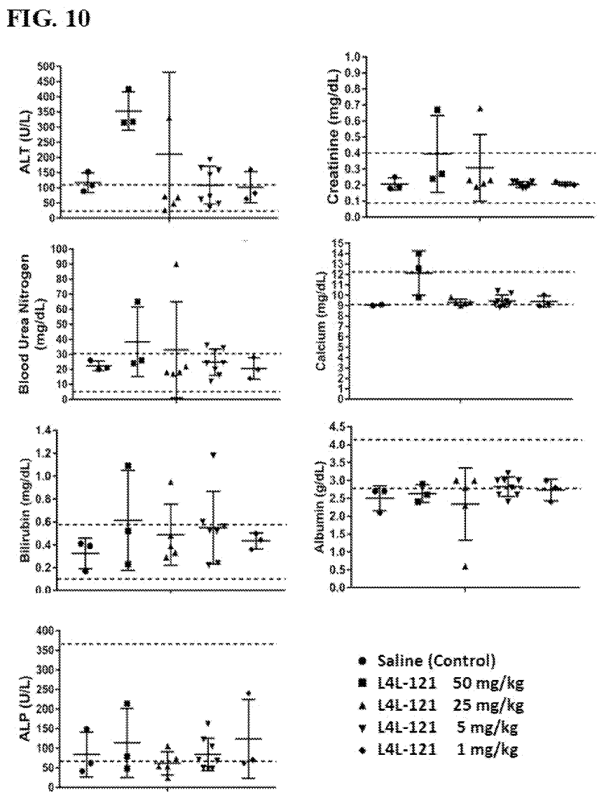

Liver function indices shown below were obtained from mice treated at 1, 5, 25 and 50 mg/kg, as well as from the untreated controls. As can be seen in FIG. 10, there was an apparent elevation in calcium levels with the 50 mg/kg dose. Further, at 50 mg/kg there was an elevation in alanine aminotransferase as seen in FIG. 10. Creatinine levels were within normal range for mice treated with 1 and 5 mg/kg of L4L-121 as well as mice treated with saline plus imipenem. Only one mouse was outside of normal range in the each of the 25 and 50 mg/kg treatment groups. Bilirubin and blood urea nitrogen levels were within normal range for mice treated with 1 mg/kg of L4L-121 and mice treated with saline plus imipenem. With increasing doses of L4L-121, a trend towards increase in these indices was noted, although the mean bilirubin levels in both the 5 and 25 mg/kg treatment groups remained within the normal range for mice, as did the mean blood urea nitrogen levels in the 5 mg/kg treatment group. The mean albumin levels were within normal range for the 1 and 5 mg/kg treatment groups and decreased albumin levels were observed in the 50 and 25 mg/kg treatment groups, as well as in the saline plus imipenem group.

### Example 4—Pharmacokinetics

A pharmacokinetic study was performed in mice where a dose of 50 mg/kg of L4L-121 was administered as a single agent and compared to a 50 mg/kg single dose of free drug trans sodium crocetinate. Blood was collected following treatment administration, per the schedule described in Table below.

TABLE 3
L4L-121 pharmacokinetic study: Treatment assignment

| | | Number | | | | Blood |
|---|---|---|---|---|---|---|
| | | of | | Route of | Dose | Collection |
| Group | Sex | Mice | Treatment | Administration | mg/kg | Timepoint |
| 1 | F | 3 | Free | Intravenous | 50 | 5 min, |
| | | | Drug | | | 30 min, 1 hr, |
| | | | TSC | | | 2 hr, 6 hr. |
| 2 | F | 3 | Free | Intravenous | 50 | 15 min, |
| | | | Drug | | | 1 hr, 2 hr, |
| | | | TSC | | | 4 hr, 8 hr. |
| 3 | F | 3 | L4L-121 | Intravenous | 50 | 5 min, 1 hr, |
| | | | | | | 2 hr, 4 hr, |
| | | | | | | 8 hr, 24 hr. |

The following pharmacokinetic parameters were then estimated:
Half-life that determines the length of the treatment effect
Area under the curve (AUC) that reflects the actual body exposure to treatment after administration of a dose of the treatment
Cₘₐₓ

TABLE 4
L4L-121 pharmacokinetic parameters

| | | | | | Plasma Exposure (fold |
|---|---|---|---|---|---|
| | | | | | increase compared to |
| | | T½ | AUC | Cₘₐₓ | free drug TSC) |
| | | (h) | (mg/ml*h) | (mg/ml) | NCA analysis |
| Group | Free Drug | 0.35 | 0.21 | 0.36 | NA |
| 1 | TSC | | | | |
| Group | Free Drug | 0.47 | 0.26 | NA | NA |
| 2 | TSC | | | | |
| Group | L4L-121 | 5.12 | 8.36 | 1.26 | 40 |
| 3 | | | | | |

As can be seen from the table above, L4L-121 formulation results in a significant improvement of half-life over free drug TSC (5-hours vs less than 0.5 hours) and approximately 40-fold improvement in exposure (AUC) of total plasma trans crocetin levels.

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

The disclosure of each of U.S. Provisional Application entitled METHODS OF SYNTHESIZING CAROTENOIDS filed Apr. 9, 2020, and Intl. Publ. No. WO2019213538, is herein incorporated by reference in its entirety.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

1. An aqueous solution comprising an ionizable carotenoid salt having the formula:
Q-trans-crocetin-Q or Q-trans-norbixin-Q
wherein,
(c)
Q is a monovalent counterion; and
polyethylene glycol (PEG) having a molecular weight of 200-700 Da;
wherein the ionizable carotenoid salt concentration is 0.1 mg/mL to 15 mg/mL, and the PEG concentration is 0.1% to 20% (w/w).

2.-8. (canceled)

9. The aqueous solution according to claim 1, wherein the ionizable carotenoid salt concentration is 0.5 mg/mL to 10 mg/mL, 1 mg/mL to 8 mg/mL, 1.5 mg/mL to 6 mg/mL, 2 mg/mL to 5 mg/mL, 2 mg/mL, 3 mg/ml, 4 mg/mL, or 5 mg/mL.

10. The aqueous solution according to claim 1, wherein the monovalent counterion (Q) is NH₄⁺, Na⁺, Li⁺, K⁺, or a monovalent organic cation such as protonated amine.

11. (canceled)

12. The aqueous solution according to claim 1, which comprises sodium trans-crocetinate (STC);

13. The aqueous solution according to claim 1, which comprises potassium trans-crocetinate (KTC), sodium trans-norbixinate (MTN), or potassium trans-norbixinate (KTN).

14. The aqueous solution according to claim 1, wherein the PEG has an average molecular weight between 200-600 Da, 300-500 Da, or 350-450 Da, (e.g., 400 Da).

15. The aqueous solution according to claim 1, wherein the PEG is PEG-200, PEG-300, PEG-400, PEG-500, or PEG-600.

16. The aqueous solution according to claim 1, which comprises PEG-400.

17. The aqueous solution according to claim 1, wherein the PEG concentration is 0.5% to 15%, 1% to 10%, 2% to 9%, 3% to 8%, or 5% to 7% (w/w).

18. The aqueous solution according to claim 1, wherein the PEG has an average molecular weight between 200-600 Da and the ionizable carotenoid salt to PEG ratio is 1:1-300; 1:1-100; 1-200; 1:1-50; 1:1-40, 1:5-30; 1:20 or 1:10-25 (w/w).

19.-21. (canceled)

22. The aqueous solution according to claim 1, which has a pH of 6-10, or any range therein between, wherein the aqueous solution comprises a buffer having a pKA within 1 unit of the pH of the solution and the buffer is at a concentration of 1-200 mM, or any range therein between.

23. (canceled)

24. The aqueous solution according to claim 22, which comprises a buffer selected from: glycine, gly-gly, sodium bicarbonate, sodium phosphate, tricine, bicine, EPPS (HEPPS), HEPBS, TABS, AMPD, and sodium borate.

25. The aqueous solution according to claim 1, which further comprises a tonicity controlling agent such as sodium chloride, mannitol, sorbitol, xylitol, dextrose, maltose, glucose, lactose or sucrose at a concentration of
(a) 0.05 mM to 100 mM, 0.75 mM to 75 mM, 1 mM to 50 mM, 5 mM to 40 mM, 7 mM to 30 mM, or 10 mM to 20 mM; or
(b) 0.05% to 20%, 0.1% to 15%, 0.5% to 10%, 0.75% to 8%, or 1% to 6% (w/w).

26.-28. (canceled)

29. The aqueous solution according to claim 1, wherein the ionizable carotenoid salt is trans-crocetin at a concentration of 1 mg/mL to 5 mg/mL (e.g., 1 mg/mL, 2 mg/mL, 3 mg/mL, 4 mg/mL, or 5 mg/mL), the PEG has an average molecular weight between 200-600 Da and the PEG concentration is 1% to 10% (e.g., 5% to 7%) (w/w).

30.-33. (canceled)

34. The aqueous solution according to claim 1, which further comprises a liposome encapsulating an ionizable carotenoid salt having the formula:
Z-trans-crocetin-Z or Z-trans-norbixin-Z
wherein,
Z is a multivalent counterion.

35.-40. (canceled)

41. The aqueous solution according to claim 34, wherein the multivalent counterion (Z) is a bivalent cation.

42. The aqueous solution according to claim 34, wherein the multivalent counterion (Z) is at least one bivalent cation selected from Ca²⁺, Mg²⁺, Zn²⁺, Cu²⁺, Co²⁺, and a bivalent organic cation.

43. The aqueous solution of claim 34, wherein the liposome encapsulates calcium trans-crocetinate (CTC).

44.-45. (canceled)

46. A pharmaceutical composition comprising the aqueous solution according to claim 1.

47.-48. (canceled)

49. A method of treating or preventing a disease or condition in a subject needing such treatment or prevention, the method comprising administering an effective amount of the pharmaceutical composition of claim 46 to the subject.

50.-92. (canceled)

93. A method of preparing a loaded liposomal pharmaceutical composition comprising:
(a) mixing the aqueous solution according to claim 1 with a liposomal solution containing liposomes in a weak acid salt of a multivalent metal; and
(b) maintaining the mixture of (a) for a sufficient time to load carotenoid into liposomes.

94. The method of claim 93, wherein the weak acid is an organic acid and the multivalent metal is a bivalent metal.

95. The method of claim 94, wherein the weak acid is an organic acid selected from acetic acid, gluconic acid, tartaric acid, glutamic acid, citric acid, formic acid, and glycinic acid and the bivalent metal is selected from Ca²⁺, Mg²⁺, Zn²⁺, Cu²⁺, and Co²⁺.

96.-97. (canceled)

98. An aqueous solution comprising a liposome prepared according to the method of claim 93.

99. (canceled)

100. A pharmaceutical composition comprising the aqueous solution according to claim 34.

101.-108. (canceled)

109. A method for treating or preventing a disease or condition in a subject needing such treatment or prevention, the method comprising administering an effective amount of the pharmaceutical composition of claim 100 to the subject.

110. The method of claim 49, wherein (1) the disease or condition is characterized by ischemia or hypoxia; (2) the disease or condition is a lung disease or a condition associated with a lung disease; (3) the disease or condition is acute respiratory distress syndrome (ARDS); (4) the disease or condition is a cardiovascular disease or a condition associated with a cardiovascular disease; (5) the disease or condition is a heart attack or stroke, or a condition associated with a heart attack or stroke; (6) the disease or condition is associated with nitric oxide deficiency; (7) the disease or condition is shock or a condition associated with shock; (8) the disease or condition is an infection or a condition associated with an infection; (9) the disease or condition is endotoxemia or a condition associated with endotoxemia; (10) the disease or condition is bacteremia; (11) the disease or condition is sepsis; or a condition associated with sepsis, or wherein the subject is at risk of developing sepsis or a condition associated with sepsis; (12) the disease or condition is inflammation or a condition associated with inflammation; (13) the disease or condition is an autoimmune disorder; (14) the disease or condition is sclerosis or a condition associated with sclerosis; (15) the disease or condition is inflammatory bowel disease or a condition associated with inflammatory bowel disease; (16) the disease or condition is a metabolic disease or a condition associated with a metabolic disease; (17) the disease or condition is insulin resistance or a condition associated with insulin resistance; (18) the disease or condition is diabetes or a condition associated with diabetes; (19) the disease or condition is type 2 diabetes or a condition associated with type 2 diabetes; (20) the disease or condition is liver disease or a condition associated with a liver disease; or (21) the disease or condition is kidney disease or a condition associated with kidney disease.

111. The method of claim 110, wherein (1) the disease or condition characterized by ischemia or hypoxia is tissue hypoperfusion, ischemic-reperfusion injury, transient cerebral ischemia, cerebral ischemia-reperfusion, ischemic stroke, hemorrhagic stroke, traumatic brain injury, migraine, gastrointestinal ischemia, kidney disease, pulmonary embolism, acute respiratory failure, neonatal respiratory distress syndrome, an obstetric emergency to reduce perinatal comorbidity, myocardial infarction, acute limb or mesenteric ischemia, cardiac cirrhosis, chronic peripheral vascular disease, congestive heart failure, atherosclerotic stenosis, anemia, thrombosis, or embolism, wherein the subject has experienced a traumatic injury, or wherein the subject is undergoing or will undergo surgery; (2) the disease or condition is pulmonary fibrosis, pulmonary hemorrhage, lung injury, lung cancer, chronic obstructive pulmonary disease (COPD), or another respiratory disorder; (3) the disease or condition is a coronary artery disease such as myocardial infarction, sudden cardiac death, cardiorespiratory arrest, hypertension, pulmonary arterial hypertension, atherosclerosis, Takayasu's arthritis, and post-cardiac arrest syndrome (PCAS), chronic venous insufficiency, heart disease, congestive heart failure, occlusive arterial disease, Raynaud's disease, peripheral vascular disease, or another vasculopathy such as Buerger's disease, or a chronic skin ulcer; (4) the disease or condition associated with nitric oxide deficiency is sickle cell disease, paroxysmal nocturnal hemoglobinuria (PNH), a hemolytic anemia, a thalassemia, another red blood cell disorder, a purpura such as thrombotic thrombocytic purpura (TTP), hemolytic uremic syndrome (HUS), idiopathic thrombocytopenia (ITP), another platelet disorder, a coagulation abnormality such as disseminated intravascular coagulopathy (DIC), purpura fulminans, heparin induced thrombocytopenia (HIT), hyperleukocytosis, and/or hyper viscosity syndrome; (5) the disease or condition is cardiogenic shock, hypovolemic shock, septic shock, neurogenic shock, or anaphylactic shock; (6) the disease or condition is hypoxia, tissue hypoperfusion, ischemia, ARDS, or sepsis; (7) the disease or condition is an P. aeruginosa infection, S. aureus infection, or an enterococcal infection; (8) the disease or condition is associated with a coronavirus infection; (9) the disease or condition is associated with an Ebola, Dengue or Marburg infection; (10) the disease or condition is associated with a candidiasis infection; (11) the disease or condition is a parasitic infection or a condition associated with a parasitic infection; (12) the disease or condition is low grade endotoxemic disease, or endotoxemia associated with periodontal disease, chronic alcoholism, chronic smoking, transplantation, neonatal necrotizing enterocolitis, or a neonatal ear infection; (13) the disease or condition is systemic inflammation, low-grade inflammation, acute inflammation, or a chronic inflammatory disease; (14) the disease or condition is psoriasis, cystic fibrosis, or rheumatoid arthritis; (15) the disease or condition is systemic sclerosis; (16) the disease or condition is Crohn's disease or ulcerative colitis; (17) the disease or condition is gangrene, diabetic necrosis, diabetic neuropathy, or diabetic vascular disease; (18) the liver disease is selected from cirrhosis, nonalcoholic fatty liver disease (NAFLD), non-alcoholic steatohepatitis (NASH); alcoholic liver disease, acute liver injury, and cirrhosis of the liver; or (19) the kidney disease is selected from lipopolysaccharide medication or toxin induced acute kidney injury (AKI) and end stage kidney disease.

112. A method of (a) increasing the delivery of oxygen to the cells and tissues in a subject, the method comprising administering an effective amount of the pharmaceutical composition of claim 46 to the subject.

113. The method of claim 112, wherein (1) the subject is in need of increased oxygen delivery; (2) the pharmaceutical composition is administered to increase the physical or mental performance of the subject; (3) wherein the age of the subject is 50 or older; (4) the subject is immunocompromised; (5) the subject receives chemotherapy and/or is immune-suppressed; (6) the subject is a burn victim; and/or (7) the subject is critically ill.

114. The method of claim 109, wherein (1) the disease or condition is characterized by ischemia or hypoxia; (2) the disease or condition is a lung disease or a condition associated with a lung disease; (3) the disease or condition is acute respiratory distress syndrome (ARDS); (4) the disease or condition is a cardiovascular disease or a condition associated with a cardiovascular disease; (5) the disease or condition is a heart attack or stroke, or a condition associated with a heart attack or stroke; (6) the disease or condition is associated with nitric oxide deficiency; (7) the disease or condition is shock or a condition associated with shock; (8) the disease or condition is an infection or a condition associated with an infection; (9) the disease or condition is endotoxemia or a condition associated with endotoxemia; (10) the disease or condition is bacteremia; (11) the disease or condition is sepsis; or a condition associated with sepsis, or wherein the subject is at risk of developing sepsis or a condition associated with sepsis; (12) the disease or condition is inflammation or a condition associated with inflammation; (13) the disease or condition is an autoimmune disorder; (14) the disease or condition is sclerosis or a condition associated with sclerosis; (15) the disease or condition is inflammatory bowel disease or a condition associated with inflammatory bowel disease; (16) the disease or condition is a metabolic disease or a condition associated with a metabolic disease; (17) the disease or condition is insulin resistance or a condition associated with insulin resistance; (18) the disease or condition is diabetes or a condition associated with diabetes; (19) the disease or condition is type 2 diabetes or a condition associated with type 2 diabetes; (20) the disease or condition is liver disease or a condition associated with a liver disease; or (21) the disease or condition is kidney disease or a condition associated with kidney disease.

115. The method of claim 114, wherein (1) the disease or condition characterized by ischemia or hypoxia is tissue hypoperfusion, ischemic-reperfusion injury, transient cerebral ischemia, cerebral ischemia-reperfusion, ischemic stroke, hemorrhagic stroke, traumatic brain injury, migraine, gastrointestinal ischemia, kidney disease, pulmonary embolism, acute respiratory failure, neonatal respiratory distress syndrome, an obstetric emergency to reduce perinatal comorbidity, myocardial infarction, acute limb or mesenteric ischemia, cardiac cirrhosis, chronic peripheral vascular disease, congestive heart failure, atherosclerotic stenosis, anemia, thrombosis, or embolism, wherein the subject has experienced a traumatic injury, or wherein the subject is undergoing or will undergo surgery; (2) the disease or condition is pulmonary fibrosis, pulmonary hemorrhage, lung injury, lung cancer, chronic obstructive pulmonary disease (COPD), or another respiratory disorder; (3) the disease or condition is a coronary artery disease such as myocardial infarction, sudden cardiac death, cardiorespiratory arrest, hypertension, pulmonary arterial hypertension, atherosclerosis, Takayasu's arthritis, and post-cardiac arrest syndrome (PCAS), chronic venous insufficiency, heart disease, congestive heart failure, occlusive arterial disease, Raynaud's disease, peripheral vascular disease, or another vasculopathy such as Buerger's disease, or a chronic skin ulcer; (4) the disease or condition associated with nitric oxide deficiency is sickle cell disease, paroxysmal nocturnal hemoglobinuria (PNH), a hemolytic anemia, a thalassemia, another red blood cell disorder, a purpura such as thrombotic thrombocytic purpura (TTP), hemolytic uremic syndrome (HUS), idiopathic thrombocytopenia (ITP), another platelet disorder, a coagulation abnormality such as disseminated intravascular coagulopathy (DIC), purpura fulminans, heparin induced thrombocytopenia (HIT), hyperleukocytosis, and/or hyper viscosity syndrome; (5) the disease or condition is cardiogenic shock, hypovolemic shock, septic shock, neurogenic shock, or anaphylactic shock; (6) the disease or condition is hypoxia, tissue hypoperfusion, ischemia, ARDS, or sepsis; (7) the disease or condition is an P. aeruginosa infection, S. aureus infection, or an enterococcal infection; (8) the disease or condition is associated with a coronavirus infection; (9) the disease or condition is associated with an Ebola, Dengue or Marburg infection; (10) the disease or condition is associated with a candidiasis infection; (11) the disease or condition is a parasitic infection or a condition associated with a parasitic infection; (12) the disease or condition is low grade endotoxemic disease, or endotoxemia associated with periodontal disease, chronic alcoholism, chronic smoking, transplantation, neonatal necrotizing enterocolitis, or a neonatal ear infection; (13) the disease or condition is systemic inflammation, low-grade inflammation, acute inflammation, or a chronic inflammatory disease; (14) the disease or condition is psoriasis, cystic fibrosis, or rheumatoid arthritis; (15) the disease or condition is systemic sclerosis; (16) the disease or condition is Crohn's disease or ulcerative colitis; (17) the disease or condition is gangrene, diabetic necrosis, diabetic neuropathy, or diabetic vascular disease; (18) the liver disease is selected from cirrhosis, nonalcoholic fatty liver disease (NAFLD), non-alcoholic steatohepatitis (NASH); alcoholic liver disease, acute liver injury, and cirrhosis of the liver; or (19) the kidney disease is selected from lipopolysaccharide medication or toxin induced acute kidney injury (AKI) and end stage kidney disease.

116. A method of (a) increasing the delivery of oxygen to the cells and tissues in a subject, the method comprising administering an effective amount of the pharmaceutical composition of claim 100 to the subject.

117. The method of claim 116, wherein (1) the subject is in need of increased oxygen delivery; (2) the pharmaceutical composition is administered to increase the physical or mental performance of the subject; (3) wherein the age of the subject is 50 or older; (4) the subject is immunocompromised; (5) the subject receives chemotherapy and/or is immune-suppressed; (6) the subject is a burn victim; and/or (7) the subject is critically ill.
